(19) United States
(12) Patent Application Publication
(10) Pub. No.: US 2016/0199410 A1
(43) Pub. Date: Jul. 14, 2016

(54) Oral Rehydration Solution

(71) Applicant: Mayo Foundation for Medical Education and Research, Rochester, MN (US)

(72) Inventors: Ryan T. Hurt, Rochester, MN (US); Jithinraj Edakkanambeth Varayll, Rochester, MN (US); Joseph M. Nadeau, Oronoco, MN (US)

(21) Appl. No.: 14/991,405

(22) Filed: Jan. 8, 2016

Related U.S. Application Data
(60) Provisional application No. 62/101,809, filed on Jan. 9, 2015.

(51) Int. Cl. A61K 33/34; A61K 31/70; A61K 31/718; A61K 33/00; A61K 33/06; A61K 33/30; A61K 33/04; A61K 31/07; A61K 31/355; A61K 31/59; A61K 31/675; A61K 31/4415; A61K 31/519; A61K 31/4406; A61K 31/522; A61K 31/194

(52) U.S. Cl. CPC A61K 33/34; A61K 31/194; A61K 31/70; A61K 31/718; A61K 33/00; A61K 33/06; A61K 33/30; A61K 33/04; A61K 31/07; A61K 31/355; A61K 31/59; A61K 31/675; A61K 31/4415; A61K 31/519; A61K 31/4406; A61K 31/522

(57) ABSTRACT

Provided herein are oral rehydration compositions useful for treating a dehydrated subject. Exemplary compositions provided herein are oral rehydration compositions customized for use with children, athletes, and subjects having short bowel syndrome. Methods of using and preparing the compositions are also provided.

### CROSS-REFERENCE TO RELATED APPLICATIONS

This application claims the benefit of U.S. Provisional Application Ser. No. 62/101,809, filed Jan. 9, 2015. The disclosure of the prior application is considered part of (and is incorporated by reference in) the disclosure of this application.

### TECHNICAL FIELD

This disclosure relates to oral rehydration compositions and methods of making and using the same.

### BACKGROUND

Dehydration, or the excessive loss of body water and associated electrolytes, occurs when water loss exceeds water intake, usually due to exercise or disease. For example, in patients having short bowel syndrome or GI surgery patients, the patients lack the ability to absorb nutrients and therefore rely on parenteral nutrition to sustain life. In such patients, extreme fluid loss and dehydration is common. Correction of dehydration is generally accomplished through replenishment of water and electrolytes through oral rehydration therapy or intravenous fluid replacement. Oral rehydration therapy is less painful, less invasive, less expensive, and easier to provide. Improved oral replacement solutions having improved properties (e.g., taste) to increase patient compliance are desirable.

### SUMMARY

Dehydration can affect a wide population of subjects, including, for example, children, patients with short bowel syndrome (SBS), and athletes. In all cases, the goal of any treatment is to provide a solution that not only addresses the condition but which is also favorable to the subject such that compliance with the treatment protocol is unpleasant or unduly burdensome.

Provided herein, inter alia, is an oral rehydration composition comprising:
about 5 to about 100 mEq/L of sodium;
about 10 to about 60 g/L of a carbohydrate source;
about 5 to about 50 mEq/L of potassium;
about 0.5 to about 50 mEq/L of magnesium; and
about 1 to about 20 mg/L of zinc.

In some embodiments, the composition comprises from about 5 to about 70 mEq/L of sodium. In some embodiments, the composition comprises from about 30 to about 90 mEq/L of sodium. In some embodiments, the sodium comprises sodium tricitrate. In some embodiments, about 10 to about 80% of the sodium in the composition is sodium tricitrate.

In some embodiments, the carbohydrate source comprises one or more of sucrose, amylase resistant starch, and mixtures thereof. In some embodiments, about 10 to about 80% of the carbohydrate source is sucrose. In some embodiments, about 10 to about 80% of the carbohydrate source is amylase resistant starch. In some embodiments, about 10 to about 75% of the carbohydrate source is amylase resistant starch. In some embodiments, the amylase resistant starch comprises high amylose maize starch (HAMS).

In some embodiments, the composition comprises about 10 to about 50 mEq/L of potassium.

In some embodiments, the composition comprises about 0.5 to about 10 mEq/L of magnesium. In some embodiments, the composition comprises about 1 to about 10 mEq/L of magnesium. In some embodiments, the composition comprises about 10 to about 50 mEq/L of magnesium. In some embodiments, the magnesium is magnesium gluconate.

In some embodiments, the composition comprises about 2 to about 10 mg/L of zinc.

In some embodiments, the composition further comprises about 10 to about 200 μg/L of selenium. In some embodiments, the composition comprises about 10 to about 100 μg/L of selenium.

In some embodiments, the composition further comprises about 1 to about 10 mg/L of copper. In some embodiments, the composition further comprises about 1 to about 5 mg/L of copper.

In some embodiments, the composition further comprises one or more of vitamin A, vitamin E, vitamin D, vitamin B6, vitamin B12, folate, niacin, and caffeine.

In some embodiments, the composition comprises one or more sweeteners. In some embodiments, the one or more sweeteners are selected from the group consisting of sucralose, stevia, aspartame, agave, and mixtures of two or more thereof.

In some embodiments, upon mixture with an effective amount of water, the composition has an osmolarity of about 200 to about 250 mOsm/L.

In some embodiments, the composition comprises about 5 to about 70 mEq/L of sodium; about 10 to about 60 g/L of a carbohydrate source; about 5 to about 50 mEq/L of potassium; about 0.5 to about 10 mEq/L of magnesium; about 1 to about 20 mg/L of zinc; and one or more sweeteners.

In some embodiments, the composition comprises about 5 to about 70 mEq/L of sodium; about 10 to about 60 g/L of a carbohydrate source, wherein about 10 to about 80% of the carbohydrate source is sucrose, an amylase resistant starch, or a mixture thereof; about 5 to about 50 mEq/L of potassium; about 0.5 to about 10 mEq/L of magnesium gluconate; about 1 to about 20 mg/L of zinc; and one or more sweeteners.

In some embodiments, the composition comprises about 5 to about 70 mEq/L of sodium; about 10 to about 60 g/L of a carbohydrate source; about 5 to about 50 mEq/L of potassium; about 1 to about 10 mEq/L of magnesium; about 2 to about 10 mg/L of zinc; about 10 to about 100 μg/L of selenium; about 1 to about 5 mg/L of copper; and one or more sweeteners. In some embodiments, the composition further comprises vitamin B6, vitamin B12, folate, niacin, and caffeine.

In some embodiments, the composition comprises about 5 to about 70 mEq/L of sodium; about 10 to about 60 g/L of a carbohydrate source, wherein about 10 to about 80% of the carbohydrate source is sucrose, an amylase resistant starch, or a mixture thereof; about 5 to about 50 mEq/L of potassium; about 1 to about 10 mEq/L of magnesium; about 2 to about 10 mg/L of zinc; about 10 to about 100 μg/L of selenium; about 1 to about 5 mg/L of copper; and one or more sweeteners. In some embodiments, the composition further comprises vitamin B6, vitamin B12, folate, niacin, and caffeine.

In some embodiments, the composition comprises about 5 to about 70 mEq/L of sodium; about 10 to about 60 g/L of a carbohydrate source, wherein about 10 to about 80% of the carbohydrate source is sucrose, an amylase resistant starch, or a mixture thereof; about 5 to about 50 mEq/L of potassium; about 1 to about 10 mEq/L of magnesium; about 2 to about 10 mg/L of zinc; about 10 to about 100 μg/L of selenium; about 1 to about 5 mg/L of copper; about 0.5 to about 50 mg/L of vitamin B6; about 1 to about 50 μg/L of vitamin B12; about 100 to about 500 μg/L of folate; about 5 to about 20 mg/L of niacin; about 5 to about 50 mg/L of caffeine; and one or more sweeteners.

In some embodiments, the composition comprises about 30 to about 90 mEq/L of sodium; about 10 to about 60 g/L of a carbohydrate source; about 10 to about 50 mEq/L of potassium; about 1 to about 10 mEq/L of magnesium; about 2 to about 20 mg/L of zinc; about 10 to about 200 μg/L of selenium; about 1 to about 10 mg/L of copper; and one or more sweeteners.

In some embodiments, the composition comprises about 30 to about 90 mEq/L of sodium; about 10 to about 60 g/L of a carbohydrate source, wherein about 10 to about 80% of the carbohydrate source is sucrose, an amylase resistant starch, or a mixture thereof; about 10 to about 50 mEq/L of potassium; about 1 to about 10 mEq/L of magnesium; about 2 to about 20 mg/L of zinc; about 10 to about 200 μg/L of selenium; about 1 to about 10 mg/L of copper; and one or more sweeteners. In some embodiments, the composition further comprises vitamin A, vitamin E, and vitamin D.

In some embodiments, the composition comprises about 30 to about 90 mEq/L of sodium; about 10 to about 60 g/L of a carbohydrate source, wherein about 10 to about 80% of the carbohydrate source is sucrose, an amylase resistant starch, or a mixture thereof; about 10 to about 50 mEq/L of potassium; about 1 to about 10 mEq/L of magnesium; about 2 to about 20 mg/L of zinc; about 10 to about 200 μg/L of selenium; about 1 to about 10 mg/L of copper; about 100 to about 20000 IU of vitamin A; about 5 to about 4000 IU of vitamin E; about 200 to about 20000 IU of vitamin D; and one or more sweeteners.

The present application further provides a method for treating or preventing dehydration in a subject in need thereof, the method comprising administering to the subject a composition provided herein. In some embodiments, the subject is a child. In some embodiments, the subject is an athlete. In some embodiments, the subject has short bowel syndrome. In some embodiments, the subject has one or more of diarrhea, vomiting, a viral infection, a bacterial infection, Crohn's disease, ischemic bowel disease, and cancer.

The present application further provides a method for treating or preventing dehydration in a subject in need thereof, the method comprising:

determining the level of dehydration in the patient; and

administering a composition provided herein.

The present application further provides a method for treating or preventing dehydration in a subject in need thereof, the method comprising:

calculating an anticipated change in the hydration level in the patient at a later point; and

administering a composition provided herein prior to the later point to lessen or prevent the anticipated change.

The present application further provides a kit comprising a composition provided herein; and instructions for preparing and using the composition. In some embodiments, one or more of the components of the composition are individually packaged. In some embodiments, all of the components of the composition are individually packaged.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DETAILED DESCRIPTION

Dehydration can affect a wide population of subjects, including, for example, children, patients with short bowel syndrome (SBS), and athletes. In all cases, the goal of any treatment is to provide a solution that not only addresses the condition but which is also favorable to the subject such that compliance with the treatment protocol is unpleasant or unduly burdensome.

For example, patients having SBS lack enough surface area in the intestinal tract to absorb nutrients and are, therefore, fully dependent upon home parenteral nutrition (HPN) to sustain life. In many cases, these patients have multiple diagnoses including Crohn's disease, ischemic bowel disease, and cancer. One of the barriers in treating these patients is the extreme fluid loss and resulting dehydration that accompanies their condition. It is, therefore, often the case that SBS patients are advised to supplement the HPN formula with oral rehydration solutions (ORS) to replenish lost water and electrolytes. While daily intake of ORS is individualized (e.g., based on GI losses), the recommended intake of ORS is generally at least about 1 liter per day in addition to the HPN formula. Compliance with this recommendation, however, has been observed as a barrier to achieving adequate hydration.

Taste has been cited as one of the major influences with compliance. In addition to SBS and GI surgery patients, other patient populations, including patients (e.g., children) with acute viral gastroenteritis or suffering from dehydration from chemotherapy treatment for cancer, would benefit from improved ORS compliance. While the addition of flavoring (e.g., the addition of free simple sugars) has been explored, these components often increase the osmolarity (i.e. osmotic concentration or solute concentration) of the ORS solution. This increase in osmolarity can become detrimental to treatment by producing increasing fluid output in the patient and leading to increased dehydration.

Provided herein are oral rehydration compositions which can be combined with water and which can be used to treat and prevent dehydration in a subject in need of such treatment. The solutions provided herein have unexpected physical properties (e.g., taste, texture, etc.) that result in higher subject approval and therefore can be used to increase subject compliance as a dehydration treatment. Moreover, the compositions provided herein can be individualized based on a number of factors including the severity of the subject's dehydration, the particular levels of one or more electrolytes in the subject, the ambient weather conditions, and the expected activity level of the subject.

The oral rehydration compositions provided herein can include one or more carbohydrates (e.g., about 10 to about 60 g/L) and one or more electrolytes (e.g., sodium, potassium, and magnesium). In some embodiments, the oral rehydration composition is a solid (e.g., a powder). In some embodiments, the oral rehydration composition is a solution (i.e. an oral rehydration solution).

In some embodiments, a solid oral rehydration composition can be combined with an effective amount of a fluid (e.g., water) to prepare an oral rehydration solution. In some embodiments, the effective amount of fluid comprises from about 93 to about 99.5% of the balance of the solution, for example, from about 93 to about 99%, from about 93 to about 98%, from about 93 to about 97.5%, from about 93 to about 97%, from about 93 to about 96.5%, from about 93 to about 96%, from about 93 to about 95.5%, from about 93 to about 95%, from about 93 to about 94.5%, from about 93 to about 94%, from about 93 to about 93.5%, from about 93.5 to about 99.5%, from about 93.5 to about 99%, from about 93.5 to about 98%, from about 93.5 to about 97.5%, from about 93.5 to about 97%, from about 93.5 to about 96.5%, from about 93.5 to about 96%, from about 93.5 to about 95.5%, from about 93.5 to about 95%, from about 93.5 to about 94.5%, from about 93.5 to about 94%, from about 94 to about 99.5%, from about 94 to about 99%, from about 94 to about 98%, from about 94 to about 97.5%, from about 94 to about 97%, from about 94 to about 96.5%, from about 94 to about 96%, from about 94 to about 95.5%, from about 94 to about 95%, from about 94 to about 94.5%, from about 94.5 to about 99.5%, from about 94.5 to about 99%, from about 94.5 to about 98%, from about 94.5 to about 97.5%, from about 94.5 to about 97%, from about 94.5 to about 96.5%, from about 94.5 to about 96%, from about 94.5 to about 95.5%, from about 94.5 to about 95%, from about 95 to about 99.5%, from about 95 to about 99%, from about 95 to about 98%, from about 95 to about 97.5%, from about 95 to about 97%, from about 95 to about 96.5%, from about 95 to about 96%, from about 95 to about 95.5%, from about 95.5 to about 99.5%, from about 95.5 to about 99%, from about 95.5 to about 98%, from about 95.5 to about 97.5%, from about 95.5 to about 97%, from about 95.5 to about 96.5%, from about 95.5 to about 96%, from about 96 to about 99.5%, from about 96 to about 99%, from about 96 to about 98%, from about 96 to about 97.5%, from about 96 to about 97%, from about 96 to about 96.5%, from about 96.5 to about 99.5%, from about 96.5 to about 99%, from about 96.5 to about 98%, from about 96.5 to about 97.5%, from about 96.5 to about 97%, from about 97 to about 99.5%, from about 97 to about 99%, from about 97 to about 98%, from about 97 to about 97.5%, from about 97.5 to about 99.5%, from about 97.5 to about 99%, from about 97.5 to about 98%, from about 98 to about 99.5%, from about 98.5 to about 99%, or from about 99 to about 99.5% of the balance of the solution.

In some embodiments, the fluid comprises water. In some embodiments, the fluid is selected from the group consisting of water, deionized water, distilled water, and purified water. In some embodiments, the water is purified by a method selected from the group consisting of reverse osmosis, carbon filtering, microfiltration, ultrafiltration, ultraviolet oxidation, and electrodialysis prior to being combined with a solid oral rehydration composition provided herein. In some embodiments, the fluid is water.

### Carbohydrates

In some embodiments, an oral rehydration composition as provided herein includes a carbohydrate source, for example, sucrose, fructose, maltose, lactose, glucose, galactose, starch, amylase resistant starch, high amylose maize starch (HAMS), dextrose, rich starch (e.g., rich flour or rice syrup), maltodextran, and mixtures thereof. In some embodiments, the carbohydrate source comprises one or more of sucrose, fructose, glucose, starch, amylase resistant starch, high amylose maize starch (HAMS), dextrose, rich starch (e.g., rich flour or rice syrup), maltodextran, and mixtures thereof. In some embodiments, the carbohydrate source comprises one or more of sucrose, amylase resistant starch, and mixtures thereof.

In some embodiments, the oral rehydration composition comprises about 5 to about 70 g/L of a carbohydrate source, for example, from about 5 to about 60 g/L, from about 5 to about 50 g/L, from about 5 to about 40 g/L, from about 5 to about 30 g/L, from about 5 to about 20 g/L, from about 5 to about 10 g/L, from about 10 to about 70 g/L, from about 10 to about 60 g/L, from about 10 to about 50 g/L, from about 10 to about 40 g/L, from about 10 to about 30 g/L, from about 10 to about 20 g/L, from about 20 to about 70 g/L, from about 20 to about 60 g/L, from about 20 to about 50 g/L, from about 20 to about 40 g/L, from about 20 to about 30 g/L, from about 30 to about 70 g/L, from about 30 to about 60 g/L, from about 30 to about 50 g/L, from about 30 to about 40 g/L, from about 40 to about 70 g/L, from about 40 to about 60 g/L, from about 40 to about 50 g/L, from about 50 to about 70 g/L, from about 50 to about 60 g/L, or from about 60 to about 70 g/L of a carbohydrate source. In some embodiments, the oral rehydration composition comprises about 10 to about 60 g/L of a carbohydrate source.

In some embodiments, about 10 to about 80% of the carbohydrate source is sucrose, for example, about 10 to about 80%, about 10 to about 75%, about 10 to about 70%, about 10 to about 65%, about 10 to about 60%, about 10 to about 55%, about 10 to about 50%, about 10 to about 45%, about 10 to about 40%, about 10 to about 35%, about 10 to about 30%, about 10 to about 25%, about 10 to about 20%, about 10 to about 15%, about 20 to about 80%, about 20 to about 75%, about 20 to about 70%, about 20 to about 65%, about 20 to about 60%, about 20 to about 55%, about 20 to about 50%, about 20 to about 45%, about 20 to about 40%, about 20 to about 35%, about 20 to about 30%, about 20 to about 25%, about 30 to about 80%, about 30 to about 75%, about 30 to about 70%, about 30 to about 65%, about 30 to about 60%, about 30 to about 55%, about 30 to about 50%, about 30 to about 45%, about 30 to about 40%, about 30 to about 35%, about 40 to about 80%, about 40 to about 75%, about 40 to about 70%, about 40 to about 65%, about 40 to about 60%, about 40 to about 55%, about 40 to about 50%, about 40 to about 45%, about 50 to about 80%, about 50 to about 75%, about 50 to about 70%, about 50 to about 65%, about 50 to about 60%, about 50 to about 55%, about 60 to about 80%, about 60 to about 75%, about 60 to about 70%, about 60 to about 65%, about 70 to about 80%, or about 70 to about 75%.

In some embodiments, about 10 to about 80% of the carbohydrate source is amylase resistant starch, for example, about 10 to about 80%, about 10 to about 75%, about 10 to about 70%, about 10 to about 65%, about 10 to about 60%, about 10 to about 55%, about 10 to about 50%, about 10 to about 45%, about 10 to about 40%, about 10 to about 35%, about 10 to about 30%, about 10 to about 25%, about 10 to about 20%, about 10 to about 15%, about 20 to about 80%, about 20 to about 75%, about 20 to about 70%, about 20 to about 65%, about 20 to about 60%, about 20 to about 55%, about 20 to about 50%, about 20 to about 45%, about 20 to about 40%, about 20 to about 35%, about 20 to about 30%, about 20 to about 25%, about 30 to about 80%, about 30 to about 75%, about 30 to about 70%, about 30 to about 65%, about 30 to about 60%, about 30 to about 55%, about 30 to about 50%, about 30 to about 45%, about 30 to about 40%, about 30 to about 35%, about 40 to about 80%, about 40 to about 75%, about 40 to about 70%, about 40 to about 65%, about 40 to about 60%, about 40 to about 55%, about 40 to about 50%, about 40 to about 45%, about 50 to about 80%, about 50 to about 75%, about 50 to about 70%, about 50 to about 65%, about 50 to about 60%, about 50 to about 55%, about 60 to about 80%, about 60 to about 75%, about 60 to about 70%, about 60 to about 65%, about 70 to about 80%, or about 70 to about 75%. In some embodiments, about 10 to about 75% of the carbohydrate source is amylase resistant starch. In some embodiments, the amylase resistant starch comprises high amylose maize starch (HAMS).

### Electrolytes

In some embodiments, an oral rehydration composition as provided herein includes one or more electrolytes, for example, sodium, potassium, magnesium, calcium, chloride, hydrogen phosphate, hydrogen carbonate, hydrogen bicarbonate. In some embodiments, the composition comprises one or more electrolytes selected from the group consisting of sodium, potassium, and magnesium. In some embodiments, the composition comprises sodium, potassium, and magnesium.

In some embodiments, the oral rehydration composition comprises from about 1 to about 120 mEq/L of sodium, for example, from about 1 to about 110 mEq/L of sodium, from about 1 to about 100 mEq/L of sodium, from about 1 to about 90 mEq/L of sodium, from about 1 to about 80 mEq/L of sodium, from about 1 to about 70 mEq/L of sodium, from about 1 to about 60 mEq/L of sodium, from about 1 to about 50 mEq/L of sodium, from about 1 to about 40 mEq/L of sodium, from about 1 to about 30 mEq/L of sodium, from about 1 to about 20 mEq/L of sodium, from about 1 to about 10 mEq/L of sodium, about 5 to about 120 mEq/L of sodium, from about 5 to about 110 mEq/L of sodium, from about 5 to about 100 mEq/L of sodium, from about 5 to about 90 mEq/L of sodium, from about 5 to about 80 mEq/L of sodium, from about 5 to about 70 mEq/L of sodium, from about 5 to about 60 mEq/L of sodium, from about 5 to about 50 mEq/L of sodium, from about 5 to about 40 mEq/L of sodium, from about 5 to about 30 mEq/L of sodium, from about 5 to about 20 mEq/L of sodium, from about 5 to about 10 mEq/L of sodium, from about 10 to about 120 mEq/L of sodium, from about 10 to about 110 mEq/L of sodium, from about 10 to about 100 mEq/L of sodium, from about 10 to about 90 mEq/L of sodium, from about 10 to about 80 mEq/L of sodium, from about 10 to about 70 mEq/L of sodium, from about 10 to about 60 mEq/L of sodium, from about 10 to about 50 mEq/L of sodium, from about 10 to about 40 mEq/L of sodium, from about 10 to about 30 mEq/L of sodium, from about 10 to about 20 mEq/L of sodium, from about 20 to about 120 mEq/L of sodium, from about 20 to about 110 mEq/L of sodium, from about 20 to about 100 mEq/L of sodium, from about 20 to about 90 mEq/L of sodium, from about 20 to about 80 mEq/L of sodium, from about 20 to about 70 mEq/L of sodium, from about 20 to about 60 mEq/L of sodium, from about 20 to about 50 mEq/L of sodium, from about 20 to about 40 mEq/L of sodium, from about 20 to about 30 mEq/L of sodium, from about 30 to about 120 mEq/L of sodium, from about 30 to about 110 mEq/L of sodium, from about 30 to about 100 mEq/L of sodium, from about 30 to about 90 mEq/L of sodium, from about 30 to about 80 mEq/L of sodium, from about 30 to about 70 mEq/L of sodium, from about 30 to about 60 mEq/L of sodium, from about 30 to about 50 mEq/L of sodium, from about 30 to about 40 mEq/L of sodium, from about 40 to about 120 mEq/L of sodium, from about 40 to about 110 mEq/L of sodium, from about 40 to about 100 mEq/L of sodium, from about 40 to about 90 mEq/L of sodium, from about 40 to about 80 mEq/L of sodium, from about 40 to about 70 mEq/L of sodium, from about 40 to about 60 mEq/L of sodium, from about 40 to about 50 mEq/L of sodium, from about 50 to about 120 mEq/L of sodium, from about 50 to about 110 mEq/L of sodium, from about 50 to about 100 mEq/L of sodium, from about 50 to about 90 mEq/L of sodium, from about 50 to about 80 mEq/L of sodium, from about 50 to about 70 mEq/L of sodium, from about 50 to about 60 mEq/L of sodium, from about 60 to about 120 mEq/L of sodium, from about 60 to about 110 mEq/L of sodium, from about 60 to about 100 mEq/L of sodium, from about 60 to about 90 mEq/L of sodium, from about 60 to about 80 mEq/L of sodium, from about 60 to about 70 mEq/L of sodium, from about 70 to about 120 mEq/L of sodium, from about 70 to about 110 mEq/L of sodium, from about 70 to about 100 mEq/L of sodium, from about 70 to about 90 mEq/L of sodium, from about 70 to about 80 mEq/L of sodium, from about 80 to about 120 mEq/L of sodium, from about 80 to about 110 mEq/L of sodium, from about 80 to about 100 mEq/L of sodium, from about 80 to about 90 mEq/L of sodium, from about 90 to about 120 mEq/L of sodium, from about 90 to about 110 mEq/L of sodium, from about 90 to about 100 mEq/L of sodium. From about 100 to about 120 mEq/L of sodium, from about 100 to about 110 mEq/L of sodium, or from about 110 to about 120 mEq/L of sodium. In some embodiments, the composition comprises about 5 to about 100 mEq/L of sodium. In some embodiments, the composition comprises from about 5 to about 70 mEp/L of sodium. In some embodiments, the composition comprises from about 30 to about 90 mEq/L of sodium.

In some embodiments, the sodium comprises a sodium salt selected from the group consisting of sodium tricitrate, sodium dicitrate, sodium citrate, sodium chloride, sodium carbonate, sodium bicarbonate, sodium triphosphate, sodium hydrogen phosphate, and sodium phosphate. In some embodiments, the sodium comprises a sodium salt selected from the group consisting of sodium tricitrate and sodium bicarbonate.

In some embodiments, the sodium comprises sodium tricitrate. In some embodiments, about 10 to about 99% of the sodium in the composition is sodium tricitrate, for example, about 10 to about 95%, about 10 to about 90%, about 10 to about 80%, about 10 to about 70%, about 10 to about 60%, about 10 to about 50%, about 10 to about 40%, about 10 to about 30%, about 10 to about 20%, about 20 to about 99%, about 20 to about 95%, about 20 to about 90%, about 20 to about 80%, about 20 to about 70%, about 20 to about 60%, about 20 to about 50%, about 20 to about 40%, about 20 to about 30%, about 30 to about 99%, about 30 to about 95%, about 30 to about 90%, about 30 to about 80%, about 30 to about 70%, about 30 to about 60%, about 30 to about 50%, about 30 to about 40%, about 40 to about 99%, about 40 to about 95%, about 40 to about 90%, about 40 to about 80%, about 40 to about 70%, about 40 to about 60%, about 40 to about 50%, about 50 to about 99%, about 50 to about 95%, about 50 to about 90%, about 50 to about 80%, about 50 to about 70%, about 50 to about 60%, about 60 to about 99%, about 60 to about 95%, about 60 to about 90%, about 60 to about 80%, about 60 to about 70%, about 70 to about 99%, about 70 to about 95%, about 70 to about 90%, about 70 to about 80%, about 80 to about 99%, about 80 to about 95%, about 80 to about 90%, about 90 to about 99%, or about 90 to about 95%. In some embodiments, about 10 to about 80% of the sodium in the composition is sodium tricitrate.

In some embodiments, the oral rehydration composition comprises from about 1 to about 60 mEq/L of potassium, for example, from about 1 to about 50 mEq/L of potassium, from about 1 to about 40 mEq/L of potassium, from about 1 to about 30 mEq/L of potassium, from about 1 to about 20 mEq/L of potassium, from about 1 to about 10 mEq/L of potassium, from about 5 to about 60 mEq/L of potassium, from about 5 to about 50 mEq/L of potassium, from about 5 to about 40 mEq/L of potassium, from about 5 to about 30 mEq/L of potassium, from about 5 to about 20 mEq/L of potassium, from about 5 to about 10 mEq/L of potassium, from about 10 to about 60 mEq/L of potassium, from about 10 to about 50 mEq/L of potassium, from about 10 to about 40 mEq/L of potassium, from about 10 to about 30 mEq/L of potassium, from about 10 to about 20 mEq/L of potassium, from about 20 to about 60 mEq/L of potassium, from about 20 to about 50 mEq/L of potassium, from about 20 to about 40 mEq/L of potassium, from about 20 to about 30 mEq/L of potassium, from about 30 to about 60 mEq/L of potassium, from about 30 to about 50 mEq/L of potassium, from about 30 to about 40 mEq/L of potassium, from about 40 to about 60 mEq/L of potassium, from about 40 to about 50 mEq/L of potassium, or from about 50 to about 60 mEq/L of potassium. In some embodiments, the composition comprises about 5 to about 50 mEq/L of potassium. In some embodiments, the composition comprises about 10 to about 50 mEq/L of potassium.

In some embodiments, the potassium comprises a potassium salt selected from the group consisting of potassium citrate, potassium chloride, potassium carbonate, potassium bicarbonate, tripotassium phosphate, dipotassium phosphate, and potassium phosphate.

In some embodiments, the oral rehydration composition comprises from about 0.1 to about 60 mEq/L of magnesium, for example, from about 0.1 to about 50 mEq/L of magnesium, from about 0.1 to about 40 mEq/L of magnesium, from about 0.1 to about 30 mEq/L of magnesium, from about 0.1 to about 20 mEq/L of magnesium, from about 0.1 to about 10 mEq/L of magnesium, from about 0.1 to about 5 mEq/L of magnesium, from about 0.1 to about 1 mEq/L of magnesium, from about 0.5 to about 60 mEq/L of magnesium, from about 0.5 to about 50 mEq/L of magnesium, from about 0.5 to about 40 mEq/L of magnesium, from about 0.5 to about 30 mEq/L of magnesium, from about 0.5 to about 20 mEq/L of magnesium, from about 0.5 to about 10 mEq/L of magnesium, from about 0.5 to about 5 mEq/L of magnesium, from about 0.5 to about 1 mEq/L of magnesium, from about 1 to about 60 mEq/L of magnesium, from about 1 to about 50 mEq/L of magnesium, from about 1 to about 40 mEq/L of magnesium, from about 1 to about 30 mEq/L of magnesium, from about 1 to about 20 mEq/L of magnesium, from about 1 to about 10 mEq/L of magnesium, from about 1 to about 5 mEq/L of magnesium, from about 5 to about 60 mEq/L of magnesium, from about 5 to about 50 mEq/L of magnesium, from about 5 to about 40 mEq/L of magnesium, from about 5 to about 30 mEq/L of magnesium, from about 5 to about 20 mEq/L of magnesium, from about 5 to about 10 mEq/L of magnesium, from about 10 to about 60 mEq/L of magnesium, from about 10 to about 50 mEq/L of magnesium, from about 10 to about 40 mEq/L of magnesium, from about 10 to about 30 mEq/L of magnesium, from about 10 to about 20 mEq/L of magnesium, from about 20 to about 60 mEq/L of magnesium, from about 20 to about 50 mEq/L of magnesium, from about 20 to about 40 mEq/L of magnesium, from about 20 to about 30 mEq/L of magnesium, from about 30 to about 60 mEq/L of magnesium, from about 30 to about 50 mEq/L of magnesium, from about 30 to about 40 mEq/L of magnesium, from about 40 to about 60 mEq/L of magnesium, from about 40 to about 50 mEq/L of magnesium, or from about 50 to about 60 mEq/L of magnesium. In some embodiments, the composition comprises about 0.5 to about 50 mEq/L of magnesium. In some embodiments, the composition comprises about 0.5 to about 10 mEq/L of magnesium. In some embodiments, the composition comprises about 1 to about 10 mEq/L of magnesium. In some embodiments, the composition comprises about 10 to about 50 mEq/L of magnesium.

In some embodiments, the magnesium comprises a magnesium salt selected from the group consisting of magnesium gluconate, magnesium citrate, magnesium chloride, magnesium carbonate, magnesium bicarbonate, magnesium phosphate tribasic, dimagnesium phosphate, and monomagnesium phosphate. In some embodiments, the magnesium is magnesium gluconate.

### Minerals

In some embodiments, an oral rehydration composition as provided herein includes one or more minerals, for example, zinc, copper, iron, manganese, molybdenum, selenium, and chromium. In some embodiments, the composition comprises zinc. In some embodiments, the composition comprises zinc or copper. In some embodiments, the composition comprises zinc, copper, or selenium. In some embodiments, the composition comprises one or more of zinc, copper, and selenium. In some embodiments, the composition comprises two or more of zinc, copper, and selenium. In some embodiments, the composition comprises zinc, copper, and selenium. In some embodiments, the composition can be an inorganic salt (e.g., a mineral sulfate, chloride, oxide, or selenite). In some embodiments, the composition comprises an inorganic salt selected from the group consisting of zinc sulfate, zinc chloride, zinc oxide, zinc gluconate, zinc stearate, copper (II) sulfate, copper (II) acetate, copper (II) chloride, copper gluconate, copper (II) oxide, sodium selenite, and potassium selenite.

In some embodiments, the composition comprises about 0.5 to about 40 mg/L of zinc, for example, from about 0.5 to about 40 mg/L of zinc, from about 0.5 to about 30 mg/L of zinc, from about 0.5 to about 20 mg/L of zinc, from about 0.5 to about 10 mg/L of zinc, from about 0.5 to about 5 mg/L of zinc, from about 0.5 to about 1 mg/L of zinc, from about 1 to about 40 mg/L of zinc, from about 1 to about 30 mg/L of zinc, from about 1 to about 20 mg/L of zinc, from about 1 to about 10 mg/L of zinc, from about 1 to about 5 mg/L of zinc, from about 10 to about 40 mg/L of zinc, from about 10 to about 30 mg/L of zinc, from about 10 to about 20 mg/L of zinc, from about 20 to about 40 mg/L of zinc, from about 20 to about 30 mg/L of zinc, or from about 30 to about 40 mg/L of zinc. In some embodiments, the composition comprises about 1 to about 20 mg/L of zinc. In some embodiments, the composition comprises about 2 to about 20 mg/L of zinc.

In some embodiments, the composition comprises about 1 to about 220 μg/L of selenium, for example, from about 1 to about 220 μg/L of selenium, from about 1 to about 200 μg/L of selenium, from about 1 to about 180 μg/L of selenium, from about 1 to about 160 μg/L of selenium, from about 1 to about 140 μg/L of selenium, from about 1 to about 120 μg/L of selenium, from about 1 to about 100 μg/L of selenium, from about 1 to about 80 μg/L of selenium, from about 1 to about 60 μg/L of selenium, from about 1 to about 40 μg/L of selenium, from about 1 to about 20 μg/L of selenium, from about 1 to about 10 μg/L of selenium, from about 10 to about 220 μg/L of selenium, from about 10 to about 200 μg/L of selenium, from about 10 to about 180 μg/L of selenium, from about 10 to about 160 μg/L of selenium, from about 10 to about 140 μg/L of selenium, from about 10 to about 120 μg/L of selenium, from about 10 to about 100 μg/L of selenium, from about 10 to about 80 μg/L of selenium, from about 10 to about 60 μg/L of selenium, from about 10 to about 40 μg/L of selenium, from about 10 to about 20 μg/L of selenium, from about 20 to about 220 μg/L of selenium, from about 20 to about 200 μg/L of selenium, from about 20 to about 180 μg/L of selenium, from about 20 to about 160 μg/L of selenium, from about 20 to about 140 μg/L of selenium, from about 20 to about 120 μg/L of selenium, from about 20 to about 100 μg/L of selenium, from about 20 to about 80 μg/L of selenium, from about 20 to about 60 μg/L of selenium, from about 20 to about 40 μg/L of selenium, from about 20 to about 30 μg/L of selenium, from about 40 to about 220 μg/L of selenium, from about 40 to about 200 μg/L of selenium, from about 40 to about 180 μg/L of selenium, from about 40 to about 160 μg/L of selenium, from about 40 to about 140 μg/L of selenium, from about 40 to about 120 μg/L of selenium, from about 40 to about 100 μg/L of selenium, from about 40 to about 80 μg/L of selenium, from about 40 to about 60 μg/L of selenium, from about 40 to about 50 μg/L of selenium, from about 60 to about 220 μg/L of selenium, from about 60 to about 200 μg/L of selenium, from about 60 to about 180 μg/L of selenium, from about 60 to about 160 μg/L of selenium, from about 60 to about 140 μg/L of selenium, from about 60 to about 120 μg/L of selenium, from about 60 to about 100 μg/L of selenium, from about 60 to about 80 μg/L of selenium, from about 60 to about 70 μg/L of selenium, from about 80 to about 220 μg/L of selenium, from about 80 to about 200 μg/L of selenium, from about 80 to about 180 μg/L of selenium, from about 80 to about 160 μg/L of selenium, from about 80 to about 140 μg/L of selenium, from about 80 to about 120 μg/L of selenium, from about 80 to about 100 μg/L of selenium, from about 80 to about 90 μg/L of selenium, from about 100 to about 220 μg/L of selenium, from about 100 to about 200 μg/L of selenium, from about 100 to about 180 μg/L of selenium, from about 100 to about 160 μg/L of selenium, from about 100 to about 140 μg/L of selenium, from about 100 to about 120 μg/L of selenium, from about 100 to about 110 μg/L of selenium, from about 120 to about 220 μg/L of selenium, from about 120 to about 200 μg/L of selenium, from about 120 to about 180 μg/L of selenium, from about 120 to about 160 μg/L of selenium, from about 120 to about 140 μg/L of selenium, from about 120 to about 130 μg/L of selenium, from about 140 to about 220 μg/L of selenium, from about 140 to about 200 μg/L of selenium, from about 140 to about 180 μg/L of selenium, from about 140 to about 160 μg/L of selenium, from about 140 to about 150 μg/L of selenium, from about 160 to about 220 μg/L of selenium, from about 160 to about 200 μg/L of selenium, from about 160 to about 180 μg/L of selenium, from about 120 to about 170 μg/L of selenium, from about 180 to about 220 μg/L of selenium, from about 180 to about 200 μg/L of selenium, from about 180 to about 190 μg/L of selenium, from about 200 to about 220 μg/L of selenium, or from about 200 to about 210 μg/L of selenium. In some embodiments, the composition comprises about 10 to about 200 μg/L of selenium. In some embodiments, the composition comprises about 10 to about 100 μg/L of selenium.

In some embodiments, the composition comprises about 0.5 to about 20 mg/L of copper, for example, from about 0.5 to about 15 mg/L of copper, from about 0.5 to about 10 mg/L of copper, from about 0.5 to about 5 mg/L of copper, from about 0.5 to about 1 mg/L of copper, from about 1 to about 20 mg/L of copper, from about 1 to about 15 mg/L of copper, from about 1 to about 10 mg/L of copper, from about 1 to about 5 mg/L of copper, from about 10 to about 20 mg/L of copper, from about 10 to about 15 mg/L of copper, or from about 15 to about 20 mg/L of copper. In some embodiments, the composition comprises about 1 to about 10 mg/L of copper. In some embodiments, the composition comprises about 1 to about 5 mg/L of copper.

### Vitamins

In some embodiments, an oral rehydration composition as provided herein includes one or more vitamins, for example, vitamin A, vitamin E, vitamin D, vitamin B1, vitamin B2, vitamin B5, vitamin B6, vitamin B12, biotin, vitamin C, choline, vitamin K, folate, and niacin. In some embodiments, the vitamin is a fat soluble vitamin. In some embodiments, the vitamin is a fat soluble vitamin in water soluble form. In some embodiments, the one or more vitamins is selected from the group consisting of vitamin A, vitamin E, vitamin D, vitamin B6, vitamin B12, folate, and niacin.

In some embodiments, the oral rehydration composition as provided herein comprises from about 0.1 to about 60 mg/L of vitamin B6, for example, from about 0.1 to about 50 mg/L of vitamin B6, from about 0.1 to about 40 mg/L of vitamin B6, from about 0.1 to about 30 mg/L of vitamin B6, from about 0.1 to about 20 mg/L of vitamin B6, from about 0.1 to about 10 mg/L of vitamin B6, from about 0.1 to about 5 mg/L of vitamin B6, from about 0.1 to about 1 mg/L of vitamin B6, from about 0.5 to about 60 mg/L of vitamin B6, from about 0.5 to about 50 mg/L of vitamin B6, from about 0.5 to about 40 mg/L of vitamin B6, from about 0.5 to about 30 mg/L of vitamin B6, from about 0.5 to about 20 mg/L of vitamin B6, from about 0.5 to about 10 mg/L of vitamin B6, from about 0.5 to about 5 mg/L of vitamin B6, from about 0.5 to about 1 mg/L of vitamin B6, from about 1 to about 60 mg/L of vitamin B6, from about 1 to about 50 mg/L of vitamin B6, from about 1 to about 40 mg/L of vitamin B6, from about 1 to about 30 mg/L of vitamin B6, from about 1 to about 20 mg/L of vitamin B6, from about 1 to about 10 mg/L of vitamin B6, from about 1 to about 5 mg/L of vitamin B6, from about 5 to about 60 mg/L of vitamin B6, from about 5 to about 50 mg/L of vitamin B6, from about 5 to about 40 mg/L of vitamin B6, from about 5 to about 30 mg/L of vitamin B6, from about 5 to about 20 mg/L of vitamin B6, from about 5 to about 10 mg/L of vitamin B6, from about 10 to about 60 mg/L of vitamin B6, from about 10 to about 50 mg/L of vitamin B6, from about 10 to about 40 mg/L of vitamin B6, from about 10 to about 30 mg/L of vitamin B6, from about 10 to about 20 mg/L of vitamin B6, from about 20 to about 60 mg/L of vitamin B6, from about 20 to about 50 mg/L of vitamin B6, from about 20 to about 40 mg/L of vitamin B6, from about 20 to about 30 mg/L of vitamin B6, from about 30 to about 60 mg/L of vitamin B6, from about 30 to about 50 mg/L of vitamin B6, from about 30 to about 40 mg/L of vitamin B6, from about 40 to about 60 mg/L of vitamin B6, from about 40 to about 50 mg/L of vitamin B6, or from about 50 to about 60 mg/L of vitamin B6. In some embodiments, the composition comprises about 0.5 to about 50 mg/L of vitamin B6.

In some embodiments, the oral rehydration composition comprises from about 0.1 to about 60 μg/L of vitamin B12, for example, from about 0.1 to about 50 μg/L of vitamin B12, from about 0.1 to about 40 μg/L of vitamin B12, from about 0.1 to about 30 μg/L of vitamin B12, from about 0.1 to about 20 μg/L of vitamin B12, from about 0.1 to about 10 μg/L of vitamin B12, from about 0.1 to about 5 μg/L of vitamin B12, from about 0.1 to about 1 μg/L of vitamin B12, from about 0.5 to about 60 μg/L of vitamin B12, from about 0.5 to about 50 μg/L of vitamin B12, from about 0.5 to about 40 μg/L of vitamin B12, from about 0.5 to about 30 μg/L of vitamin B12, from about 0.5 to about 20 μg/L of vitamin B12, from about 0.5 to about 10 μg/L of vitamin B12, from about 0.5 to about 5 μg/L of vitamin B12, from about 0.5 to about 1 μg/L of vitamin B12, from about 1 to about 60 μg/L of vitamin B12, from about 1 to about 50 μg/L of vitamin B12, from about 1 to about 40 μg/L of vitamin B12, from about 1 to about 30 μg/L of vitamin B12, from about 1 to about 20 μg/L of vitamin B12, from about 1 to about 10 μg/L of vitamin B12, from about 1 to about 5 μg/L of vitamin B12, from about 5 to about 60 μg/L of vitamin B12, from about 5 to about 50 μg/L of vitamin B12, from about 5 to about 40 μg/L of vitamin B12, from about 5 to about 30 μg/L of vitamin B12, from about 5 to about 20 μg/L of vitamin B12, from about 5 to about 10 μg/L of vitamin B12, from about 10 to about 60 μg/L of vitamin B12, from about 10 to about 50 μg/L of vitamin B12, from about 10 to about 40 μg/L of vitamin B12, from about 10 to about 30 μg/L of vitamin B12, from about 10 to about 20 μg/L of vitamin B12, from about 20 to about 60 μg/L of vitamin B12, from about 20 to about 50 μg/L of vitamin B12, from about 20 to about 40 μg/L of vitamin B12, from about 20 to about 30 μg/L of vitamin B12, from about 30 to about 60 μg/L of vitamin B12, from about 30 to about 50 μg/L of vitamin B12, from about 30 to about 40 μg/L of vitamin B12, from about 40 to about 60 μg/L of vitamin B12, from about 40 to about 50 μg/L of vitamin B12, or from about 50 to about 60 μg/L of vitamin B12. In some embodiments, the oral rehydration composition comprises from about 1 to about 50 μg/L of vitamin B12.

In some embodiments, the oral rehydration composition comprises from about 50 to about 600 μg/L of folate, for example, from about 50 to about 500 μg/L of folate, from about 50 to about 400 μg/L of folate, from about 50 to about 300 μg/L of folate, from about 50 to about 200 μg/L of folate, from about 50 to about 100 μg/L of folate, from about 50 to about 75 μg/L of folate, from about 75 to about 600 μg/L of folate, from about 75 to about 500 μg/L of folate, from about 75 to about 400 μg/L of folate, from about 75 to about 300 μg/L of folate, from about 75 to about 200 μg/L of folate, from about 75 to about 100 μg/L of folate, from about 100 to about 600 μg/L of folate, from about 100 to about 500 μg/L of folate, from about 100 to about 400 μg/L of folate, from about 100 to about 300 μg/L of folate, from about 100 to about 200 μg/L of folate, from about 200 to about 600 μg/L of folate, from about 200 to about 500 μg/L of folate, from about 200 to about 400 μg/L of folate, from about 200 to about 300 μg/L of folate, from about 300 to about 600 μg/L of folate, from about 300 to about 500 μg/L of folate, from about 300 to about 400 μg/L of folate, from about 400 to about 600 μg/L of folate, from about 400 to about 500 μg/L of folate, or from about 500 to about 600 μg/L of folate. In some embodiments, the composition comprises about 100 to about 500 μg/L of folate.

In some embodiments, the oral rehydration composition comprises from about 1 to about 40 mg/L of niacin, for example, from about 1 to about 30 mg/L of niacin, from about 1 to about 20 mg/L of niacin, from about 1 to about 10 mg/L of niacin, from about 1 to about 5 mg/L of niacin, from about 5 to about 40 mg/L of niacin, from about 5 to about 30 mg/L of niacin, from about 5 to about 20 mg/L of niacin, from about 5 to about 10 mg/L of niacin, from about 10 to about 40 mg/L of niacin, from about 10 to about 30 mg/L of niacin, from about 10 to about 20 mg/L of niacin, from about 20 to about 40 mg/L of niacin, from about 20 to about 30 mg/L of niacin, or from about 30 to about 40 mg/L of niacin. In some embodiments, the oral rehydration composition comprises about 5 to about 20 mg/L of niacin.

In some embodiments, the oral rehydration composition comprises about 50 to about 25000 IU of vitamin A, for example, from about 50 to about 20000 IU of vitamin A, from about 50 to about 15000 IU of vitamin A, from about 50 to about 10000 IU of vitamin A, from about 50 to about 9000 IU of vitamin A, from about 50 to about 8000 IU of vitamin A, from about 50 to about 7000 IU of vitamin A, from about 50 to about 6000 IU of vitamin A, from about 50 to about 5000 IU of vitamin A, from about 50 to about 4000 IU of vitamin A, from about 50 to about 3000 IU of vitamin A, from about 50 to about 2000 IU of vitamin A, from about 50 to about 1000 IU of vitamin A, from about 50 to about 750 IU of vitamin A, from about 50 to about 500 IU of vitamin A, from about 50 to about 250 IU of vitamin A, from about 50 to about 100 IU of vitamin A, from about 75 to about 25000 IU of vitamin A, from about 75 to about 20000 IU of vitamin A, from about 75 to about 15000 IU of vitamin A, from about 75 to about 10000 IU of vitamin A, from about 75 to about 9000 IU of vitamin A, from about 75 to about 8000 IU of vitamin A, from about 75 to about 7000 IU of vitamin A, from about 75 to about 6000 IU of vitamin A, from about 75 to about 5000 IU of vitamin A, from about 75 to about 4000 IU of vitamin A, from about 75 to about 3000 IU of vitamin A, from about 75 to about 2000 IU of vitamin A, from about 75 to about 1000 IU of vitamin A, from about 75 to about 750 IU of vitamin A, from about 75 to about 500 IU of vitamin A, from about 75 to about 250 IU of vitamin A, from about 75 to about 100 IU of vitamin A, from about 100 to about 25000 IU of vitamin A, from about 100 to about 20000 IU of vitamin A, from about 100 to about 15000 IU of vitamin A, from about 100 to about 10000 IU of vitamin A, from about 100 to about 9000 IU of vitamin A, from about 100 to about 8000 IU of vitamin A, from about 100 to about 7000 IU of vitamin A, from about 100 to about 6000 IU of vitamin A, from about 100 to about 5000 IU of vitamin A, from about 100 to about 4000 IU of vitamin A, from about 100 to about 3000 IU of vitamin A, from about 100 to about 2000 IU of vitamin A, from about 100 to about 1000 IU of vitamin A, from about 100 to about 750 IU of vitamin A, from about 100 to about 500 IU of vitamin A, from about 100 to about 250 IU of vitamin A, from about 250 to about 25000 IU of vitamin A, from about 250 to about 20000 IU of vitamin A, from about 250 to about 15000 IU of vitamin A, from about 250 to about 10000 IU of vitamin A, from about 250 to about 9000 IU of vitamin A, from about 250 to about 8000 IU of vitamin A, from about 250 to about 7000 IU of vitamin A, from about 250 to about 6000 IU of vitamin A, from about 250 to about 5000 IU of vitamin A, from about 250 to about 4000 IU of vitamin A, from about 250 to about 3000 IU of vitamin A, from about 250 to about 2000 IU of vitamin A, from about 250 to about 1000 IU of vitamin A, from about 250 to about 750 IU of vitamin A, from about 250 to about 500 IU of vitamin A, from about 500 to about 25000 IU of vitamin A, from about 500 to about 20000 IU of vitamin A, from about 500 to about 15000 IU of vitamin A, from about 500 to about 10000 IU of vitamin A, from about 500 to about 9000 IU of vitamin A, from about 500 to about 8000 IU of vitamin A, from about 500 to about 7000 IU of vitamin A, from about 500 to about 6000 IU of vitamin A, from about 500 to about 5000 IU of vitamin A, from about 500 to about 4000 IU of vitamin A, from about 500 to about 3000 IU of vitamin A, from about 500 to about 2000 IU of vitamin A, from about 500 to about 1000 IU of vitamin A, from about 500 to about 750 IU of vitamin A, from about 750 to about 25000 IU of vitamin A, from about 750 to about 20000 IU of vitamin A, from about 750 to about 15000 IU of vitamin A, from about 750 to about 10000 IU of vitamin A, from about 750 to about 9000 IU of vitamin A, from about 750 to about 8000 IU of vitamin A, from about 750 to about 7000 IU of vitamin A, from about 750 to about 6000 IU of vitamin A, from about 750 to about 5000 IU of vitamin A, from about 750 to about 4000 IU of vitamin A, from about 750 to about 3000 IU of vitamin A, from about 750 to about 2000 IU of vitamin A, from about 750 to about 1000 IU of vitamin A, from about 1000 to about 25000 IU of vitamin A, from about 1000 to about 20000 IU of vitamin A, from about 1000 to about 15000 IU of vitamin A, from about 1000 to about 10000 IU of vitamin A, from about 1000 to about 9000 IU of vitamin A, from about 1000 to about 8000 IU of vitamin A, from about 1000 to about 7000 IU of vitamin A, from about 1000 to about 6000 IU of vitamin A, from about 1000 to about 5000 IU of vitamin A, from about 1000 to about 4000 IU of vitamin A, from about 1000 to about 3000 IU of vitamin A, from about 1000 to about 2000 IU of vitamin A, from about 2000 to about 25000 IU of vitamin A, from about 2000 to about 20000 IU of vitamin A, from about 2000 to about 15000 IU of vitamin A, from about 2000 to about 10000 IU of vitamin A, from about 2000 to about 9000 IU of vitamin A, from about 2000 to about 8000 IU of vitamin A, from about 2000 to about 7000 IU of vitamin A, from about 2000 to about 6000 IU of vitamin A, from about 2000 to about 5000 IU of vitamin A, from about 2000 to about 4000 IU of vitamin A, from about 2000 to about 3000 IU of vitamin A, from about 3000 to about 25000 IU of vitamin A, from about 3000 to about 20000 IU of vitamin A, from about 3000 to about 15000 IU of vitamin A, from about 3000 to about 10000 IU of vitamin A, from about 3000 to about 9000 IU of vitamin A, from about 3000 to about 8000 IU of vitamin A, from about 3000 to about 7000 IU of vitamin A, from about 3000 to about 6000 IU of vitamin A, from about 3000 to about 5000 IU of vitamin A, from about 3000 to about 4000 IU of vitamin A, from about 4000 to about 25000 IU of vitamin A, from about 4000 to about 20000 IU of vitamin A, from about 4000 to about 15000 IU of vitamin A, from about 4000 to about 10000 IU of vitamin A, from about 4000 to about 9000 IU of vitamin A, from about 4000 to about 8000 IU of vitamin A, from about 4000 to about 7000 IU of vitamin A, from about 4000 to about 6000 IU of vitamin A, from about 4000 to about 5000 IU of vitamin A, from about 5000 to about 25000 IU of vitamin A, from about 5000 to about 20000 IU of vitamin A, from about 5000 to about 15000 IU of vitamin A, from about 5000 to about 10000 IU of vitamin A, from about 5000 to about 9000 IU of vitamin A, from about 5000 to about 8000 IU of vitamin A, from about 5000 to about 7000 IU of vitamin A, from about 5000 to about 6000 IU of vitamin A, from about 6000 to about 25000 IU of vitamin A, from about 6000 to about 20000 IU of vitamin A, from about 6000 to about 15000 IU of vitamin A, from about 6000 to about 10000 IU of vitamin A, from about 6000 to about 9000 IU of vitamin A, from about 6000 to about 8000 IU of vitamin A, from about 6000 to about 7000 IU of vitamin A, from about 7000 to about 25000 IU of vitamin A, from about 7000 to about 20000 IU of vitamin A, from about 7000 to about 15000 IU of vitamin A, from about 7000 to about 10000 IU of vitamin A, from about 7000 to about 9000 IU of vitamin A, from about 7000 to about 8000 IU of vitamin A, from about 8000 to about 25000 IU of vitamin A, from about 8000 to about 20000 IU of vitamin A, from about 8000 to about 15000 IU of vitamin A, from about 8000 to about 10000 IU of vitamin A, from about 8000 to about 9000 IU of vitamin A, from about 9000 to about 25000 IU of vitamin A, from about 9000 to about 20000 IU of vitamin A, from about 9000 to about 15000 IU of vitamin A, from about 9000 to about 10000 IU of vitamin A, from about 10000 to about 25000 IU of vitamin A, from about 10000 to about 20000 IU of vitamin A, from about 10000 to about 15000 IU of vitamin A, from about 15000 to about 25000 IU of vitamin A, from about 15000 to about 20000 IU of vitamin A, or from about 20000 to about 25000 IU of vitamin A. In some embodiments, the oral rehydration composition comprises about 100 to about 20000 IU of vitamin A.

In some embodiments, the oral rehydration composition comprises about 50 to about 25000 IU of vitamin D, for example, from about 50 to about 20000 IU of vitamin D, from about 50 to about 15000 IU of vitamin D, from about 50 to about 10000 IU of vitamin D, from about 50 to about 9000 IU of vitamin D, from about 50 to about 8000 IU of vitamin D, from about 50 to about 7000 IU of vitamin D, from about 50 to about 6000 IU of vitamin D, from about 50 to about 5000 IU of vitamin D, from about 50 to about 4000 IU of vitamin D, from about 50 to about 3000 IU of vitamin D, from about 50 to about 2000 IU of vitamin D, from about 50 to about 1000 IU of vitamin D, from about 50 to about 750 IU of vitamin D, from about 50 to about 500 IU of vitamin D, from about 50 to about 250 IU of vitamin D, from about 50 to about 100 IU of vitamin D, from about 75 to about 25000 IU of vitamin D, from about 75 to about 20000 IU of vitamin D, from about 75 to about 15000 IU of vitamin D, from about 75 to about 10000 IU of vitamin D, from about 75 to about 9000 IU of vitamin D, from about 75 to about 8000 IU of vitamin D, from about 75 to about 7000 IU of vitamin D, from about 75 to about 6000 IU of vitamin D, from about 75 to about 5000 IU of vitamin D, from about 75 to about 4000 IU of vitamin D, from about 75 to about 3000 IU of vitamin D, from about 75 to about 2000 IU of vitamin D, from about 75 to about 1000 IU of vitamin D, from about 75 to about 750 IU of vitamin D, from about 75 to about 500 IU of vitamin D, from about 75 to about 250 IU of vitamin D, from about 75 to about 100 IU of vitamin D, from about 100 to about 25000 IU of vitamin D, from about 100 to about 20000 IU of vitamin D, from about 100 to about 15000 IU of vitamin D, from about 100 to about 10000 IU of vitamin D, from about 100 to about 9000 IU of vitamin D, from about 100 to about 8000 IU of vitamin D, from about 100 to about 7000 IU of vitamin D, from about 100 to about 6000 IU of vitamin D, from about 100 to about 5000 IU of vitamin D, from about 100 to about 4000 IU of vitamin D, from about 100 to about 3000 IU of vitamin D, from about 100 to about 2000 IU of vitamin D, from about 100 to about 1000 IU of vitamin D, from about 100 to about 750 IU of vitamin D, from about 100 to about 500 IU of vitamin D, from about 100 to about 250 IU of vitamin D, from about 250 to about 25000 IU of vitamin D, from about 250 to about 20000 IU of vitamin D, from about 250 to about 15000 IU of vitamin D, from about 250 to about 10000 IU of vitamin D, from about 250 to about 9000 IU of vitamin D, from about 250 to about 8000 IU of vitamin D, from about 250 to about 7000 IU of vitamin D, from about 250 to about 6000 IU of vitamin D, from about 250 to about 5000 IU of vitamin D, from about 250 to about 4000 IU of vitamin D, from about 250 to about 3000 IU of vitamin D, from about 250 to about 2000 IU of vitamin D, from about 250 to about 1000 IU of vitamin D, from about 250 to about 750 IU of vitamin D, from about 250 to about 500 IU of vitamin D, from about 500 to about 25000 IU of vitamin D, from about 500 to about 20000 IU of vitamin D, from about 500 to about 15000 IU of vitamin D, from about 500 to about 10000 IU of vitamin D, from about 500 to about 9000 IU of vitamin D, from about 500 to about 8000 IU of vitamin D, from about 500 to about 7000 IU of vitamin D, from about 500 to about 6000 IU of vitamin D, from about 500 to about 5000 IU of vitamin D, from about 500 to about 4000 IU of vitamin D, from about 500 to about 3000 IU of vitamin D, from about 500 to about 2000 IU of vitamin D, from about 500 to about 1000 IU of vitamin D, from about 500 to about 750 IU of vitamin D, from about 750 to about 25000 IU of vitamin D, from about 750 to about 20000 IU of vitamin D, from about 750 to about 15000 IU of vitamin D, from about 750 to about 10000 IU of vitamin D, from about 750 to about 9000 IU of vitamin D, from about 750 to about 8000 IU of vitamin D, from about 750 to about 7000 IU of vitamin D, from about 750 to about 6000 IU of vitamin D, from about 750 to about 5000 IU of vitamin D, from about 750 to about 4000 IU of vitamin D, from about 750 to about 3000 IU of vitamin D, from about 750 to about 2000 IU of vitamin D, from about 750 to about 1000 IU of vitamin D, from about 1000 to about 25000 IU of vitamin D, from about 1000 to about 20000 IU of vitamin D, from about 1000 to about 15000 IU of vitamin D, from about 1000 to about 10000 IU of vitamin D, from about 1000 to about 9000 IU of vitamin D, from about 1000 to about 8000 IU of vitamin D, from about 1000 to about 7000 IU of vitamin D, from about 1000 to about 6000 IU of vitamin D, from about 1000 to about 5000 IU of vitamin D, from about 1000 to about 4000 IU of vitamin D, from about 1000 to about 3000 IU of vitamin D, from about 1000 to about 2000 IU of vitamin D, from about 2000 to about 25000 IU of vitamin D, from about 2000 to about 20000 IU of vitamin D, from about 2000 to about 15000 IU of vitamin D, from about 2000 to about 10000 IU of vitamin D, from about 2000 to about 9000 IU of vitamin D, from about 2000 to about 8000 IU of vitamin D, from about 2000 to about 7000 IU of vitamin D, from about 2000 to about 6000 IU of vitamin D, from about 2000 to about 5000 IU of vitamin D, from about 2000 to about 4000 IU of vitamin D, from about 2000 to about 3000 IU of vitamin D, from about 3000 to about 25000 IU of vitamin D, from about 3000 to about 20000 IU of vitamin D, from about 3000 to about 15000 IU of vitamin D, from about 3000 to about 10000 IU of vitamin D, from about 3000 to about 9000 IU of vitamin D, from about 3000 to about 8000 IU of vitamin D, from about 3000 to about 7000 IU of vitamin D, from about 3000 to about 6000 IU of vitamin D, from about 3000 to about 5000 IU of vitamin D, from about 3000 to about 4000 IU of vitamin D, from about 4000 to about 25000 IU of vitamin D, from about 4000 to about 20000 IU of vitamin D, from about 4000 to about 15000 IU of vitamin D, from about 4000 to about 10000 IU of vitamin D, from about 4000 to about 9000 IU of vitamin D, from about 4000 to about 8000 IU of vitamin D, from about 4000 to about 7000 IU of vitamin D, from about 4000 to about 6000 IU of vitamin D, from about 4000 to about 5000 IU of vitamin D, from about 5000 to about 25000 IU of vitamin D, from about 5000 to about 20000 IU of vitamin D, from about 5000 to about 15000 IU of vitamin D, from about 5000 to about 10000 IU of vitamin D, from about 5000 to about 9000 IU of vitamin D, from about 5000 to about 8000 IU of vitamin D, from about 5000 to about 7000 IU of vitamin D, from about 5000 to about 6000 IU of vitamin D, from about 6000 to about 25000 IU of vitamin D, from about 6000 to about 20000 IU of vitamin D, from about 6000 to about 15000 IU of vitamin D, from about 6000 to about 10000 IU of vitamin D, from about 6000 to about 9000 IU of vitamin D, from about 6000 to about 8000 IU of vitamin D, from about 6000 to about 7000 IU of vitamin D, from about 7000 to about 25000 IU of vitamin D, from about 7000 to about 20000 IU of vitamin D, from about 7000 to about 15000 IU of vitamin D, from about 7000 to about 10000 IU of vitamin D, from about 7000 to about 9000 IU of vitamin D, from about 7000 to about 8000 IU of vitamin D, from about 8000 to about 25000 IU of vitamin D, from about 8000 to about 20000 IU of vitamin D, from about 8000 to about 15000 IU of vitamin D, from about 8000 to about 10000 IU of vitamin D, from about 8000 to about 9000 IU of vitamin D, from about 9000 to about 25000 IU of vitamin D, from about 9000 to about 20000 IU of vitamin D, from about 9000 to about 15000 IU of vitamin D, from about 9000 to about 10000 IU of vitamin D, from about 10000 to about 25000 IU of vitamin D, from about 10000 to about 20000 IU of vitamin D, from about 10000 to about 15000 IU of vitamin D, from about 15000 to about 25000 IU of vitamin D, from about 15000 to about 20000 IU of vitamin D, or from about 20000 to about 25000 IU of vitamin D. In some embodiments, the oral rehydration composition comprises about 200 to about 20000 IU of vitamin D.

In some embodiments, the oral rehydration composition comprises from about 1 to about 5000 IU of vitamin E, for example, from about 1 to about 4000 IU of vitamin E, from about 1 to about 3000 IU of vitamin E, from about 1 to about 2000 IU of vitamin E, from about 1 to about 1000 IU of vitamin E, from about 1 to about 500 IU of vitamin E, from about 1 to about 250 IU of vitamin E, from about 1 to about 100 IU of vitamin E, from about 1 to about 50 IU of vitamin E, from about 5 to about 5000 IU of vitamin E, from about 5 to about 4000 IU of vitamin E, from about 5 to about 3000 IU of vitamin E, from about 5 to about 2000 IU of vitamin E, from about 5 to about 1000 IU of vitamin E, from about 5 to about 500 IU of vitamin E, from about 5 to about 250 IU of vitamin E, from about 5 to about 100 IU of vitamin E, from about 5 to about 50 IU of vitamin E, from about 50 to about 5000 IU of vitamin E, from about 50 to about 4000 IU of vitamin E, from about 50 to about 3000 IU of vitamin E, from about 50 to about 2000 IU of vitamin E, from about 50 to about 1000 IU of vitamin E, from about 50 to about 500 IU of vitamin E, from about 50 to about 250 IU of vitamin E, from about 50 to about 100 IU of vitamin E, from about 100 to about 5000 IU of vitamin E, from about 100 to about 4000 IU of vitamin E, from about 100 to about 3000 IU of vitamin E, from about 100 to about 2000 IU of vitamin E, from about 100 to about 1000 IU of vitamin E, from about 100 to about 500 IU of vitamin E, from about 100 to about 250 IU of vitamin E, from about 250 to about 5000 IU of vitamin E, from about 250 to about 4000 IU of vitamin E, from about 250 to about 3000 IU of vitamin E, from about 250 to about 2000 IU of vitamin E, from about 250 to about 1000 IU of vitamin E, from about 250 to about 500 IU of vitamin E, from about 500 to about 5000 IU of vitamin E, from about 500 to about 4000 IU of vitamin E, from about 500 to about 3000 IU of vitamin E, from about 500 to about 2000 IU of vitamin E, from about 500 to about 1000 IU of vitamin E, from about 1000 to about 5000 IU of vitamin E, from about 1000 to about 4000 IU of vitamin E, from about 1000 to about 3000 IU of vitamin E, from about 1000 to about 2000 IU of vitamin E, from about 2000 to about 5000 IU of vitamin E, from about 2000 to about 4000 IU of vitamin E, from about 2000 to about 3000 IU of vitamin E, from about 3000 to about 5000 IU of vitamin E, from about 3000 to about 4000 IU of vitamin E, or from about 4000 to about 5000 IU of vitamin E. In some embodiments, the oral rehydration composition comprises about 5 to about 4000 IU of vitamin E.

### Sweeteners

In some embodiments, an oral rehydration composition as provided herein includes one or more sweeteners, for example, sucralose, stevia, aspartame, agave, acacia syrup, butterscotch, calcium citrate, cocoa, cyclamate, glycyrrhiza (licorice syrup), honey, lemon extract, lemon oil, lime extract, lime oil, monoammonium glyrrhizinate (MagnaSweet®), mannitol, maple, neohesperidine DC, neotame, Prosweet® Powder, saccharin, sorbitol, acesulfame postassium, tagatose, xylitol, or any combination thereof. In some embodiments, the one or more sweetener is selected from the group consisting of sucralose, stevia, aspartame, and agave, and mixtures of two or more thereof.

### Additional Components

In some embodiments, an oral rehydration composition as provided herein includes one or more additional components (i.e., a coloring agent, a preserving agent, or flavoring agent), for example, caffeine, green tea extract, maltol, annatto extract, dehydrated beets (beet powder), canthaxanthin, caramel, β-carotene, grape skin extract (enocianina), fruit juice, vegetable juice, carrot oil, riboflavin, tomato lycopene extract, tomato lycopene concentrate, inulin, soluble fiber, insoluble fiber, probiotics, β-hydroxy-β-methylbutyrate (HMB), L-glutamine, L-arginine, L-leucine, L-isoleucine, and L-valine.

In some embodiments, an oral rehydration composition as provided herein includes one or more flavoring agents. In some embodiments, the one or more flavoring agents is selected from the group consisting of alitame, anise, anise flavor, apple flavor, banana flavor, Bavarian cream, Bavarian cream flavor, berry flavor, black currant, butterscotch flavor, camphor, caramel, caramel flavor, cherry flavor, cherry cream flavor, chocolate, chocolate flavor, cinnamon, cinnamon flavor, bubble gum flavor, citrus flavor, citrus punch flavor, citrus cream flavor, cotton candy flavor, cocoa flavor, cola, cola flavor, cool cherry flavor, cool citrus flavor, dextrose, eucalyptus flavor, eugenol, fructose, fruit punch, fruit punch flavor, grape flavor, grapefruit flavor, honey, isomalt, lemon flavor, licorice flavor, lime flavor, lemon cream flavor, maltol, maple flavor, marshmallow flavor, menthol, mint cream flavor, mixed berry flavor, orange flavor, pear flavor, peach flavor, peppermint flavor, peppermint cream flavor, raspberry flavor, root beer flavor, rum flavor, saccharin, safrole, sorbitol, spearmint flavor, spearmint cream flavor, strawberry flavor, strawberry cream flavor, talin, sylitol, Swiss cream flavor, tangerine flavor, thaumatin, tutti frutti flavor, vanilla flavor, walnut flavor, watermelon flavor, wild cherry flavor, wintergreen flavor, or a mixture thereof (e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof).

In some embodiments, the oral rehydration composition comprises from about 1 to about 60 mg/L of caffeine, for example, from about 1 to about 50 mg/L of caffeine, from about 1 to about 40 mg/L of caffeine, from about 1 to about 30 mg/L of caffeine, from about 1 to about 20 mg/L of caffeine, from about 1 to about 10 mg/L of caffeine, from about 1 to about 5 mg/L of caffeine, from about 5 to about 60 mg/L of caffeine, from about 5 to about 50 mg/L of caffeine, from about 5 to about 40 mg/L of caffeine, from about 5 to about 30 mg/L of caffeine, from about 5 to about 20 mg/L of caffeine, from about 5 to about 10 mg/L of caffeine, from about 10 to about 60 mg/L of caffeine, from about 10 to about 50 mg/L of caffeine, from about 10 to about 40 mg/L of caffeine, from about 10 to about 30 mg/L of caffeine, from about 10 to about 20 mg/L of caffeine, from about 20 to about 60 mg/L of caffeine, from about 20 to about 50 mg/L of caffeine, from about 20 to about 40 mg/L of caffeine, from about 20 to about 30 mg/L of caffeine, from about 30 to about 60 mg/L of caffeine, from about 30 to about 50 mg/L of caffeine, from about 30 to about 40 mg/L of caffeine, from about 40 to about 60 mg/L of caffeine, from about 40 to about 50 mg/L of caffeine, or from about 50 to about 60 mg/L of caffeine. In some embodiments, the oral rehydration composition comprises about 50 to about 50 mg/L of caffeine.

### Specific Embodiments/Formulations

The present application provides an oral rehydration composition comprising:
about 5 to about 100 mEq/L of sodium;
about 10 to about 60 g/L of a carbohydrate source;
about 5 to about 50 mEq/L of potassium;
about 0.5 to about 50 mEq/L of magnesium; and
about 1 to about 20 mg/L of zinc.

In some embodiments, the composition comprises from about 5 to about 70 mEq/L of sodium. In some embodiments, the composition comprises from about 30 to about 90 mEq/L of sodium. In some embodiments, the sodium comprises sodium tricitrate. In some embodiments, about 10 to about 80% of the sodium in the composition is sodium tricitrate. In some embodiments, the carbohydrate source comprises one or more of sucrose, amylase resistant starch, and mixtures thereof. In some embodiments, about 10 to about 80% of the carbohydrate source is sucrose. In some embodiments, about 10 to about 80% of the carbohydrate source is amylase resistant starch. In some embodiments, about 10 to about 75% of the carbohydrate source is amylase resistant starch. In some embodiments, the amylase resistant starch comprises high amylose maize starch (HAMS). In some embodiments, the composition comprises about 10 to about 50 mEq/L of potassium. In some embodiments, the composition comprises about 0.5 to about 10 mEq/L of magnesium. In some embodiments, the composition comprises about 1 to about 10 mEq/L of magnesium. In some embodiments, the composition comprises about 10 to about 50 mEq/L of magnesium. In some embodiments, the magnesium is magnesium gluconate. In some embodiments, the composition comprises about 2 to about 10 mg/L of zinc. In some embodiments, the composition further comprises about 10 to about 200 μg/L of selenium. In some embodiments, the composition comprises about 10 to about 100 μg/L of selenium. In some embodiments, the composition further comprises about 1 to about 10 mg/L of copper. In some embodiments, the composition further comprises about 1 to about 5 mg/L of copper. In some embodiments, the composition further comprises one or more of vitamin A, vitamin E, vitamin D, vitamin B6, vitamin B12, folate, niacin, and caffeine. In some embodiments, the composition comprises one or more sweeteners. In some embodiments, the one or more sweeteners are selected from the group consisting of sucralose, stevia, aspartame, agave, and mixtures of two or more thereof. In some embodiments, upon mixture with an effective amount of water, the composition has an osmolarity of about 200 to about 250 mOsm/L.

In some embodiments, the composition comprises:
about 5 to about 70 mEq/L of sodium;
about 10 to about 60 g/L of a carbohydrate source;
about 5 to about 50 mEq/L of potassium;
about 0.5 to about 10 mEq/L of magnesium;
about 1 to about 20 mg/L of zinc; and
one or more sweeteners.

In some embodiments, the composition comprises:
about 5 to about 70 mEq/L of sodium;
about 10 to about 60 g/L of a carbohydrate source, wherein about 10 to about 80% of the carbohydrate source is sucrose, an amylase resistant starch, or a mixture thereof;
about 5 to about 50 mEq/L of potassium;
about 0.5 to about 10 mEq/L of magnesium gluconate;
about 1 to about 20 mg/L of zinc; and
one or more sweeteners.

In some embodiments, the composition comprises:
about 5 to about 70 mEq/L of sodium;
about 10 to about 60 g/L of a carbohydrate source;
about 5 to about 50 mEq/L of potassium;
about 1 to about 10 mEq/L of magnesium;
about 2 to about 10 mg/L of zinc;
about 10 to about 100 μg/L of selenium;
about 1 to about 5 mg/L of copper; and
one or more sweeteners.

In some embodiments, the composition comprises:
about 5 to about 70 mEq/L of sodium;
about 10 to about 60 g/L of a carbohydrate source, wherein about 10 to about 80% of the carbohydrate source is sucrose, an amylase resistant starch, or a mixture thereof;
about 5 to about 50 mEq/L of potassium;
about 1 to about 10 mEq/L of magnesium;
about 2 to about 10 mg/L of zinc;
about 10 to about 100 μg/L of selenium;
about 1 to about 5 mg/L of copper; and
one or more sweeteners. In some embodiments, the composition further comprises vitamin B6, vitamin B12, folate, niacin, and caffeine.

In some embodiments, the composition comprises:
about 5 to about 70 mEq/L of sodium;
about 10 to about 60 g/L of a carbohydrate source, wherein about 10 to about 80% of the carbohydrate source is sucrose, an amylase resistant starch, or a mixture thereof;
about 5 to about 50 mEq/L of potassium;
about 1 to about 10 mEq/L of magnesium;
about 2 to about 10 mg/L of zinc;
about 10 to about 100 μg/L of selenium;
about 1 to about 5 mg/L of copper;
about 0.5 to about 50 mg/L of vitamin B6;
about 1 to about 50 μg/L of vitamin B12;
about 100 to about 500 μg/L of folate;
about 5 to about 20 mg/L of niacin;
about 5 to about 50 mg/L of caffeine; and
one or more sweeteners.

In some embodiments, the composition comprises:
about 30 to about 90 mEq/L of sodium;
about 10 to about 60 g/L of a carbohydrate source;
about 10 to about 50 mEq/L of potassium;
about 1 to about 10 mEq/L of magnesium;
about 2 to about 20 mg/L of zinc;
about 10 to about 200 μg/L of selenium;
about 1 to about 10 mg/L of copper; and
one or more sweeteners.

In some embodiments, the composition comprises:
about 30 to about 90 mEq/L of sodium;
about 10 to about 60 g/L of a carbohydrate source, wherein about 10 to about 80% of the carbohydrate source is sucrose, an amylase resistant starch, or a mixture thereof;
about 10 to about 50 mEq/L of potassium;
about 1 to about 10 mEq/L of magnesium;
about 2 to about 20 mg/L of zinc;
about 10 to about 200 μg/L of selenium;
about 1 to about 10 mg/L of copper; and
one or more sweeteners. In some embodiments, the composition further comprises vitamin A, vitamin E, and vitamin D.

In some embodiments, the composition comprises:
about 30 to about 90 mEq/L of sodium;
about 10 to about 60 g/L of a carbohydrate source, wherein about 10 to about 80% of the carbohydrate source is sucrose, an amylase resistant starch, or a mixture thereof;
about 10 to about 50 mEq/L of potassium;
about 1 to about 10 mEq/L of magnesium;
about 2 to about 20 mg/L of zinc;
about 10 to about 200 μg/L of selenium;
about 1 to about 10 mg/L of copper;
about 100 to about 20000 IU of vitamin A;
about 5 to about 4000 IU of vitamin E;
about 200 to about 20000 IU of vitamin D; and
one or more sweeteners.

### Osmolarity

In some embodiments, an oral rehydration composition as provided herein, upon mixture with an effective amount of water, has an osmolarity of about 100 to about 300 mOsm/L, for example, about 100 to about 280 mOsm/L, about 100 to about 260 mOsm/L, about 100 to about 250 mOsm/L, about 100 to about 240 mOsm/L, about 100 to about 220 mOsm/L, about 100 to about 200 mOsm/L, about 100 to about 180 mOsm/L, about 100 to about 160 mOsm/L, about 100 to about 140 mOsm/L, about 100 to about 120 mOsm/L, about 120 to about 300 mOsm/L, about 120 to about 280 mOsm/L, about 120 to about 260 mOsm/L, about 120 to about 250 mOsm/L, about 120 to about 240 mOsm/L, about 120 to about 220 mOsm/L, about 120 to about 200 mOsm/L, about 120 to about 180 mOsm/L, about 120 to about 160 mOsm/L, about 120 to about 140 mOsm/L, about 140 to about 300 mOsm/L, about 140 to about 280 mOsm/L, about 140 to about 260 mOsm/L, about 140 to about 250 mOsm/L, about 140 to about 240 mOsm/L, about 140 to about 220 mOsm/L, about 140 to about 200 mOsm/L, about 140 to about 180 mOsm/L, about 140 to about 160 mOsm/L, about 160 to about 300 mOsm/L, about 160 to about 280 mOsm/L, about 160 to about 260 mOsm/L, about 160 to about 250 mOsm/L, about 160 to about 240 mOsm/L, about 160 to about 220 mOsm/L, about 160 to about 200 mOsm/L, about 160 to about 180 mOsm/L, about 180 to about 300 mOsm/L, about 180 to about 280 mOsm/L, about 180 to about 260 mOsm/L, about 180 to about 250 mOsm/L, about 180 to about 240 mOsm/L, about 180 to about 220 mOsm/L, about 180 to about 200 mOsm/L, about 200 to about 300 mOsm/L, about 200 to about 280 mOsm/L, about 200 to about 260 mOsm/L, about 200 to about 250 mOsm/L, about 200 to about 240 mOsm/L, about 200 to about 220 mOsm/L, about 220 to about 300 mOsm/L, about 220 to about 280 mOsm/L, about 220 to about 260 mOsm/L, about 220 to about 250 mOsm/L, about 220 to about 240 mOsm/L, about 240 to about 300 mOsm/L, about 240 to about 280 mOsm/L, about 240 to about 260 mOsm/L, about 240 to about 250 mOsm/L, about 250 to about 300 mOsm/L, about 250 to about 280 mOsm/L, about 250 to about 260 mOsm/L, about 260 to about 300 mOsm/L, about 260 to about 280 mOsm/L, or about 280 to about 300 mOsm/L. In some embodiments, the composition, upon mixture with an effective amount of water, has an osmolarity of about 200 to about 250 mOsm/L.

### Methods of Use

Provided herein are methods for treating or preventing dehydration in a subject in need thereof. In some embodiments, the dehydration is classified as mild dehydration (e.g., 3%-5% dehydration). In some embodiments, the dehydration is classified as moderate dehydration (e.g., 6%-9% dehydration). In some embodiments, the dehydration is classified as severe dehydration (e.g., greater than 10% dehydration). In some embodiments, the dehydration is transient dehydration.

As used herein, the term “subject,” refers to a mammal, including humans. In some embodiments, the subject is a human. In some embodiments, the subject is a child. In some embodiments, the subject is an athlete. In some embodiments, the subject has short bowl syndrome. In some embodiments, the subject has one or more additional disorder. In some embodiments, the additional disorder is selected from the group consisting of diarrhea, vomiting, diarrhea, vomiting, a viral infection, a bacterial infection, Crohn's disease, ischemic bowel disease, and cancer. In some embodiments, the subject has one or more of diarrhea, vomiting, diarrhea, vomiting, a viral infection, a bacterial infection, Crohn's disease, ischemic bowel disease, and cancer. In some embodiments, the method comprises administering to the subject an effective amount of an oral composition provided herein.

In some embodiments, the oral rehydration composition provided herein can be customized for a subject prior to treating the subject. In some embodiments, the method for treating or preventing dehydration in a subject in need thereof comprises:

a) determining the level of dehydration in a subject; and

b) administering an oral rehydration composition provided herein.

In some embodiments, the method for treating or preventing dehydration in a subject in need thereof comprises:

a) calculating an anticipated change in the hydration level in the patient at a later point; and

b) administering an oral rehydration composition provided herein prior to the later point to lessen or prevent the anticipated change.

In some embodiments, the composition is customized for a child. In some embodiments, the composition is customized for an athlete. In some embodiments, the composition is customized for a subject having short bowl syndrome. In some embodiments, the composition is customized for a child, athlete, or subject having short bowel syndrome, wherein the child, athlete, or subject having short bowel syndrome have one or more of diarrhea, vomiting, diarrhea, vomiting, a viral infection, a bacterial infection, Crohn's disease, ischemic bowel disease, and cancer.

In some embodiments, determining the level of dehydration in a subject comprises determining one or more factors of dehydration. In some embodiments, the one or more factors of dehydration is selected from the group consisting of the electrolyte level of a subject, relative humidity, the ambient temperature, blood loss or hypotension of the patient due to physical trauma, hyperthermia, shock, vomiting, burns, lacrimation, exposure to radiation, use of drugs (e.g., methamphetamine, amphetamine, caffeine and other stimulants), excessive consumption of alcoholic beverages, malnutrition, electrolyte disturbance, fasting, severe hyperglycemia (e.g., severe hyperglycemia associated with diabetes mellitus), glycosuria, uremia, diabetes insipidus, Type 1 diabetes, type 2 diabetes, Sjogren's Syndrome, and pressure ulcers. In some embodiments, the factor of dehydration is physical activity or diarrhea (e.g., acute diarrhea). In some embodiments, the factor of dehydration is prolonged physical activity (e.g., cycling, running, hiking). In some embodiments, the factor of dehydration is infection from a pathogen, for example, Vibrio cholerae, Campylobater jejuni, Giardia lamblia, Clostridium botulinum, Listeria monocytogenes, Staphylococcus aureus, Salmonella, Shigella, and Campylobacter enteritis. In some embodiments, the factor of dehydration results from a bowel disorder. In some embodiments, the factor of dehydration results from an inflammatory bowel disorder. In some embodiments, the factor of dehydration results from ulcerative colitis, Crohn's disease, IBS, or a combination thereof. In some embodiments, the factor of dehydration results from a bowel preparation (e.g., preparation for a colonoscopy). In some embodiments, the factor of dehydration results from cystic fibrosis. In some embodiments, the factor of dehydration results from AIDS and/or AIDS Wasting syndrome. In some embodiments, the factor of dehydration results from hyperemesis gravidarum. In some embodiments, the factor of dehydration results from third spacing (e.g., following a surgical procedure such as liposuction, or due to the presence of burns on the body). In some embodiments, the factor of dehydration results from ascites, accumulation of fluid at a burn site, pleural effusion, or a combination thereof

### Methods of Preparation

The present application further provides methods of preparing the oral rehydration compositions provided herein. In some embodiments, the method of preparing the oral rehydration composition comprises:

i) blending one or more independently selected sweetener with one or more independently selected carbohydrate sources to provide a first mixture;

ii) blending one or more independently selected salts (e.g., sodium chloride, potassium chloride) and trisodium citrate to provide a second mixture;

iii) adding one or more independently selected bicarbonate salts (e.g., sodium bicarbonate, potassium bicarbonate) and one or more independently selected additional components (e.g., a coloring agent, a preserving agent, or flavoring agent as defined herein) to the second mixture to provide a third mixture; and

iv) blending the first mixture with the third mixture to provide an oral rehydration composition.

In some embodiments, the method of preparing the oral rehydration composition comprises:

i) blending one or more independently selected sweeteners (e.g., sucralose, stevia, aspartame, agave, or mixtures of two or more thereof) and one or more independently selected carbohydrate sources (e.g., sucrose, amylase resistant starch, and mixtures thereof) to provide a first mixture;

ii) blending one or more independently selected salts (e.g., sodium chloride, potassium chloride) and trisodium citrate to provide a second mixture;

iii) blending one or more independently selected bicarbonate salts (e.g., sodium bicarbonate, potassium bicarbonate) and one or more independently selected additional components (e.g., a coloring agent, a preserving agent, or flavoring agent as defined herein) into the second mixture to provide a third mixture; and

iv) blending the first mixture with the third mixture to provide an oral rehydration composition.

In some embodiments, the blending of step i) is performed from about 4 to about 10 minutes, for example, from about 4 to about 9 minutes, from about 4 to about 8 minutes, from about 4 to about 7 minutes, from about 4 to about 6 minutes, from about 4 to about 5 minutes, from about 5 to about 10 minutes, from about 5 to about 9 minutes, from about 5 to about 8 minutes, from about 5 to about 7 minutes, from about 5 to about 6 minutes, from about 6 to about 10 minutes, from about 6 to about 9 minutes, from about 6 to about 8 minutes, from about 6 to about 7 minutes, from about 7 to about 10 minutes, from about 7 to about 9 minutes, from about 7 to about 8 minutes, from about 8 to about 10 minutes, from about 8 to about 9 minutes, or from about 9 to about 10 minutes. In some embodiments, the blending of step i) is performed from about 6 to about 8 minutes.

In some embodiments, the blending of step ii) is performed from about 2 to about 10 minutes, for example, from about 2 to about 9 minutes, from about 2 to about 8 minutes, from about 2 to about 7 minutes, from about 2 to about 6 minutes, from about 2 to about 5 minutes, from about 2 to about 4 minutes, from about 2 to about 3 minutes, from about 3 to about 10 minutes, from about 3 to about 9 minutes, from about 3 to about 8 minutes, from about 3 to about 7 minutes, from about 3 to about 6 minutes, from about 3 to about 5 minutes, from about 3 to about 4 minutes, from about 4 to about 10 minutes, from about 4 to about 9 minutes, from about 4 to about 8 minutes, from about 4 to about 7 minutes, from about 4 to about 6 minutes, from about 4 to about 5 minutes, from about 5 to about 10 minutes, from about 5 to about 9 minutes, from about 5 to about 8 minutes, from about 5 to about 7 minutes, from about 5 to about 6 minutes, from about 6 to about 10 minutes, from about 6 to about 9 minutes, from about 6 to about 8 minutes, from about 6 to about 7 minutes, from about 7 to about 10 minutes, from about 7 to about 9 minutes, from about 7 to about 8 minutes, from about 8 to about 10 minutes, from about 8 to about 9 minutes, or from about 9 to about 10 minutes. In some embodiments, the blending of step ii) is performed from about 4 to about 6 minutes.

In some embodiments, the blending of step iii) is performed from about 2 to about 10 minutes, for example, from about 2 to about 9 minutes, from about 2 to about 8 minutes, from about 2 to about 7 minutes, from about 2 to about 6 minutes, from about 2 to about 5 minutes, from about 2 to about 4 minutes, from about 2 to about 3 minutes, from about 3 to about 10 minutes, from about 3 to about 9 minutes, from about 3 to about 8 minutes, from about 3 to about 7 minutes, from about 3 to about 6 minutes, from about 3 to about 5 minutes, from about 3 to about 4 minutes, from about 4 to about 10 minutes, from about 4 to about 9 minutes, from about 4 to about 8 minutes, from about 4 to about 7 minutes, from about 4 to about 6 minutes, from about 4 to about 5 minutes, from about 5 to about 10 minutes, from about 5 to about 9 minutes, from about 5 to about 8 minutes, from about 5 to about 7 minutes, from about 5 to about 6 minutes, from about 6 to about 10 minutes, from about 6 to about 9 minutes, from about 6 to about 8 minutes, from about 6 to about 7 minutes, from about 7 to about 10 minutes, from about 7 to about 9 minutes, from about 7 to about 8 minutes, from about 8 to about 10 minutes, from about 8 to about 9 minutes, or from about 9 to about 10 minutes. In some embodiments, the blending of step iii) is performed from about 4 to about 6 minutes.

In some embodiments, the blending of step iv) is performed from about 2 to about 10 minutes, for example, from about 2 to about 9 minutes, from about 2 to about 8 minutes, from about 2 to about 7 minutes, from about 2 to about 6 minutes, from about 2 to about 5 minutes, from about 2 to about 4 minutes, from about 2 to about 3 minutes, from about 3 to about 10 minutes, from about 3 to about 9 minutes, from about 3 to about 8 minutes, from about 3 to about 7 minutes, from about 3 to about 6 minutes, from about 3 to about 5 minutes, from about 3 to about 4 minutes, from about 4 to about 10 minutes, from about 4 to about 9 minutes, from about 4 to about 8 minutes, from about 4 to about 7 minutes, from about 4 to about 6 minutes, from about 4 to about 5 minutes, from about 5 to about 10 minutes, from about 5 to about 9 minutes, from about 5 to about 8 minutes, from about 5 to about 7 minutes, from about 5 to about 6 minutes, from about 6 to about 10 minutes, from about 6 to about 9 minutes, from about 6 to about 8 minutes, from about 6 to about 7 minutes, from about 7 to about 10 minutes, from about 7 to about 9 minutes, from about 7 to about 8 minutes, from about 8 to about 10 minutes, from about 8 to about 9 minutes, or from about 9 to about 10 minutes. In some embodiments, the blending of step iv) is performed for about 2 minutes, for about 3 minutes, for about 4 minutes, for about 5 minutes, for about 6 minutes, for about 7 minutes, for about 8 minutes, for about 9 minutes, or for about 10 minutes. In some embodiments, the blending of step iv) is performed for about 5 minutes.

In some embodiments, the blending of step i) is performed using an industrial grade food processor, an industrial grade blender, or an industrial grade mixer. In some embodiments, the blending of step i) is performed at high speed. In some embodiments, the blending of step i) is performed using an industrial grade food processor at high speed.

In some embodiments, the blending of step ii) is performed using an industrial grade food processor, an industrial grade blender, or an industrial grade mixer.

In some embodiments, the blending of step iii) is performed using an industrial grade food processor, an industrial grade blender, or an industrial grade mixer.

In some embodiments, the blending of step iv) is performed using an industrial grade food processor, an industrial grade blender, or an industrial grade mixer. In some embodiments, the blending of step iv) is performed using an industrial grade whisk.

In some embodiments, the blending of steps i)-iv) is performed in one or more industrial grade food processors. In some embodiments, the blending of step i) is performed in a first industrial grade food processor and the blending of steps ii) and iii) is performed in a second industrial grade food processor.

In some embodiments, the oral rehydration composition is prepared in a large batch. In some embodiments, the large batch comprises about 5 to about 200 L, for example, about 5 to about 175 L, about 5 to about 150 L, about 5 to about 125 L, about 5 to about 100 L, about 5 to about 75 L, about 5 to about 50 L, about 5 to about 25 L, about 5 to about 10 L, about 10 to about 200 L, 10 to about 175 L, about 10 to about 150 L, about 10 to about 125 L, about 10 to about 100 L, about 10 to about 75 L, about 10 to about 50 L, about 10 to about 25 L, about 25 to about 200 L, 25 to about 175 L, about 25 to about 150 L, about 25 to about 125 L, about 25 to about 100 L, about 25 to about 75 L, about 25 to about 50 L, about 50 to about 200 L, 50 to about 175 L, about 50 to about 150 L, about 50 to about 125 L, about 50 to about 100 L, about 50 to about 75 L, about 75 to about 200 L, 75 to about 175 L, about 75 to about 150 L, about 75 to about 125 L, about 75 to about 100 L, about 100 to about 200 L, 100 to about 175 L, about 100 to about 150 L, about 100 to about 125 L, about 125 to about 200 L, 125 to about 175 L, about 125 to about 150 L, about 150 to about 200 L, about 150 to about 175 L, or about 175 to about 200 L.

### Kits

The present application further provides a kit for use in treating or preventing dehydration, comprising an oral rehydration composition provided herein and instructions for preparing and using the composition. In some embodiments, the kit comprises an oral rehydration composition comprising one or more of a carbohydrate component, one or more of an electrolyte component, and one or more of a mineral component provided herein. In some embodiments, the kit further comprises one or more of a vitamin component provided herein. In some embodiments, the kit further comprises one or more of a sweetener component provided herein. In some embodiments, the kit further comprises one or more of an additional component provided herein. In some embodiments, the one or more components of the oral rehydration composition are individually packaged. In some embodiments, all of the components of the composition are individually packaged. In some embodiments, the kit comprises instructions for preparing and using the composition. In some embodiments, the instructions include recommended daily dosage amounts. In some embodiments, the oral rehydration composition is a solid dosage form and the instructions include directions for mixing the solid dosage for with a suitable amount of a suitable liquid (e.g. water). In some embodiments, the kit comprises an oral rehydration composition provided herein packaged in a single use package. In some embodiments, the kit comprises an oral rehydration composition provided herein packaged in a multi-use package.

In some embodiments, the kit is customized for use in treating or preventing dehydration in a child. In some embodiments, the kit is customized for use in treating or preventing dehydration in an athlete. In some embodiments, the kit is customized for use in treating or preventing dehydration in a subject having short bowel syndrome. In some embodiments, the kit is customized for use in treating or preventing dehydration in a child, athlete, or subject having short bowel syndrome, wherein the child, athlete, or subject having short bowel syndrome have one or more of diarrhea, vomiting, diarrhea, vomiting, a viral infection, a bacterial infection, Crohn's disease, ischemic bowel disease, and cancer.

### DEFINITIONS

As used herein, “osmolarity” or “osmotic concentration” is a measure of solute concentration and is defined as the number of osmoles (Osm) of solute per liter (L) of solution (Osm/L). Multiple compounds may contribute to the osmolarity of a solution.

As used herein, “IU” refers to the “International Unit”, a unit of measurement for the amount of substance (e.g., unit of measurement for the amount of a vitamin). As used herein, the mass equivalents of 1 IU of vitamins A, D, and E are defined as the following:

Vitamin A: 1 IU is the biological equivalent of 0.3 μg retinol, or of 0.6 μg beta-carotene.

Vitamin D: 1 IU is the biological equivalent of 0.025 μg cholecalciferol/ergocalciferol.

Vitamin E: 1 IU is the biological equivalent of about 0.667 mg d-α-tocopherol (⅔ mg exactly), or of 0.45 mg of dl-α-tocopherol acetate.

### Examples

The invention is further described in the following examples, which do not limit the scope of the invention described in the claims.

### Example 1

### Oral Rehydration Composition for Subject Having Short Bowel Syndrome

The following components and concentrations are provided for preparing an oral rehydration composition customized for use with a subject having short bowel syndrome:

| | |
|---|---|
| Sodium | 30-90 mEq/L |
| Tricitrate sodium | supplies 10-80% of sodium |
| Sucrose | 10-80% of carbohydrate source |
| Amylase resistant starch | 10-80% of carbohydrate source |
| (HAMS) | |
| Potassium | 10-50 mEq/L |
| Magnesium (as gluconate) | 1-10 mEq/L |
| Zinc | 2-20 mg/L |
| Selenium | 10-200 μg/L |
| Copper | 1-10 mg/L |
| Fat soluble vitamins (water soluble | |
| form) | |
| Vitamin A | 1000-20000 IU |
| Vitamin E | 5-4000 IU |
| Vitamin D | 200-20000 IU |
| Sweeteners | various, including sucralose, stevia, |
| | aspartame, agave |
| Desired osmolarity | 200-250 mOsm/L |

### Example 2

### Additional Oral Rehydration Compositions for Subject Having Short Bowel Syndrome

Table 1 provides components for preparing an oral rehydration composition for use with a subject who has short bowel syndrome and a) does not have a colon; and b) has an intact colon.

TABLE 1

| a) Subject without Colon | b) Subject with Intact Colon |
|---|---|
| Glucose, rice starch, non- | Amylase resistant starch + Glucose (1:1, |
| amylase resistant starch | 1:2, 2:1, 3:1, 1:3) |
| Salt | Salt |
| Salt substitute | Salt substitute |
| Trisodium citrate/Baking soda | Trisodium citrate/Baking soda |
| Vitamin B complex, Water | Vitamin B complex, Water soluble form |
| soluble form of fat soluble | of fat soluble vitamins |
| vitamins Cu, Zn, Se | Cu, Zn, Se |
| | fiber |
| One or more flavoring agent | One or more flavoring agent as defined |
| as defined herein | herein |

### Example 3

### Oral Rehydration Composition for Athletes

The following components and concentrations are provided for preparing an oral rehydration composition customized for use with an athlete:

| | |
|---|---|
| Sodium | 5-70 mEq/L |
| Tricitrate sodium | supplies 10-60% of sodium |
| Sucrose | 10-80% of carbohydrate source |
| Amylase resistant starch | 10-75% of carbohydrate source |
| (HAMS) | |
| Potassium | 5-50 mEq/L |
| Magnesium (as gluconate) | 1-10 mEq/L |
| Zinc | 2-10 mg/L |
| Selenium | 10-100 μg/L |
| Copper | 1-5 mg/L |
| Sweeteners | various, including sucralose, stevia, |
| | aspartame, agave |
| Desired osmolarity | 200-250 mOsm/L |
| Vitamin B6 | 0.5-50 mg/L |
| Vitamin B12 | 1-50 μg/L |
| Folate | 100-500 μg/L |
| Niacin | 5-20 mg/L |
| Caffeine | 5-50 mg/L |

### Example 4

### Oral Rehydration Composition for Children

The following components and concentrations are provided for preparing an oral rehydration composition customized for use with children:

| | |
|---|---|
| Sodium | 5-70 mEq/L |
| Tricitrate sodium | supplies 10-80% of sodium |
| Sucrose | 10-80% of carbohydrate source |
| Amylase resistant starch | 10-80% of carbohydrate source |
| (HAMS) | |
| Potassium | 5-50 mEq/L |
| Magnesium (as gluconate) | 0.5-10 mEq/L |
| Zinc | 1-20 mg/L |
| Sweeteners | various, including sucralose, stevia, |
| | aspartame, agave |
| Desired osmolarity | 200-250 mOsm/L |

### Example 5

### Oral Rehydration Composition for the General Public

Table 2 provides components for an oral rehydration composition for use with the general public (including e.g., athletes, children, subjects with viral gastroenteritis, and subjects having short bowel syndrome).

TABLE 2

Unique blend of starch and sugar (e.g., 1:1, 1:2, 2:1, 3:1, 1:3)
Amylase resistant starch + instant energy − Glucose
Salt
Salt substitute
Trisodium citrate
Flavoring

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An oral rehydration composition comprising:
about 5 to about 100 mEq/L of sodium;
about 10 to about 60 g/L of a carbohydrate source;
about 5 to about 50 mEq/L of potassium;
about 0.5 to about 50 mEq/L of magnesium; and
about 1 to about 20 mg/L of zinc.

2.-3. (canceled)

4. The composition of claim 1, wherein the sodium comprises sodium tricitrate.

5. (canceled)

6. The composition of claim 1, wherein the carbohydrate source comprises one or more of sucrose, amylase resistant starch, and mixtures thereof.

7.-9. (canceled)

10. The composition of claim 6, wherein the amylase resistant starch comprises high amylose maize starch (HAMS).

11.-14. (canceled)

15. The composition of claim 1, wherein the magnesium is magnesium gluconate.

16. (canceled)

17. The composition of claim 1, wherein the composition further comprises about 10 to about 200 μg/L of selenium.

18. (canceled)

19. The composition of claim 1, wherein the composition further comprises about 1 to about 10 mg/L of copper.

20. (canceled)

21. The composition of claim 1, wherein the composition further comprises one or more of vitamin A, vitamin E, vitamin D, vitamin B6, vitamin B12, folate, niacin, and caffeine.

22. The composition of claim 1, wherein the composition comprises one or more sweeteners.

23. (canceled)

24. The composition of claim 1, wherein upon mixture with an effective amount of water, the composition has an osmolarity of about 200 to about 250 mOsm/L.

25. (canceled)

26. The composition of claim 1, wherein the composition comprises:
about 5 to about 70 mEq/L of sodium;
about 10 to about 60 g/L of a carbohydrate source, wherein about 10 to about 80% of the carbohydrate source is sucrose, an amylase resistant starch, or a mixture thereof;
about 5 to about 50 mEq/L of potassium;
about 0.5 to about 10 mEq/L of magnesium gluconate;
about 1 to about 20 mg/L of zinc; and
one or more sweeteners.

27. The composition of claim 1, wherein the composition comprises:
about 5 to about 70 mEq/L of sodium;
about 10 to about 60 g/L of a carbohydrate source;
about 5 to about 50 mEq/L of potassium;
about 1 to about 10 mEq/L of magnesium;
about 2 to about 10 mg/L of zinc;
about 10 to about 100 μg/L of selenium;
about 1 to about 5 mg/L of copper; and
one or more sweeteners.

28. The composition of claim 1, wherein the composition comprises:
about 5 to about 70 mEq/L of sodium;
about 10 to about 60 g/L of a carbohydrate source, wherein about 10 to about 80% of the carbohydrate source is sucrose, an amylase resistant starch, or a mixture thereof;
about 5 to about 50 mEq/L of potassium;
about 1 to about 10 mEq/L of magnesium;
about 2 to about 10 mg/L of zinc;
about 10 to about 100 μg/L of selenium;
about 1 to about 5 mg/L of copper; and
one or more sweeteners.

29. (canceled)

30. The composition of claim 1, wherein the composition comprises:
about 5 to about 70 mEq/L of sodium;
about 10 to about 60 g/L of a carbohydrate source, wherein about 10 to about 80% of the carbohydrate source is sucrose, an amylase resistant starch, or a mixture thereof;
about 5 to about 50 mEq/L of potassium;
about 1 to about 10 mEq/L of magnesium;
about 2 to about 10 mg/L of zinc;
about 10 to about 100 μg/L of selenium;
about 1 to about 5 mg/L of copper;
about 0.5 to about 50 mg/L of vitamin B6;
about 1 to about 50 μg/L of vitamin B12;
about 100 to about 500 μg/L of folate;
about 5 to about 20 mg/L of niacin;
about 5 to about 50 mg/L of caffeine; and
one or more sweeteners.

31. The composition of claim 1, wherein the composition comprises:
about 30 to about 90 mEq/L of sodium;
about 10 to about 60 g/L of a carbohydrate source;
about 10 to about 50 mEq/L of potassium;
about 1 to about 10 mEq/L of magnesium;
about 2 to about 20 mg/L of zinc;
about 10 to about 200 μg/L of selenium;
about 1 to about 10 mg/L of copper; and
one or more sweeteners.

32. The composition of claim 1, wherein the composition comprises:
about 30 to about 90 mEq/L of sodium;
about 10 to about 60 g/L of a carbohydrate source, wherein about 10 to about 80% of the carbohydrate source is sucrose, an amylase resistant starch, or a mixture thereof;
about 10 to about 50 mEq/L of potassium;
about 1 to about 10 mEq/L of magnesium;
about 2 to about 20 mg/L of zinc;
about 10 to about 200 μg/L of selenium;
about 1 to about 10 mg/L of copper; and
one or more sweeteners.

33. (canceled)

34. The composition of claim 1, wherein the composition comprises:
about 30 to about 90 mEq/L of sodium;
about 10 to about 60 g/L of a carbohydrate source, wherein about 10 to about 80% of the carbohydrate source is sucrose, an amylase resistant starch, or a mixture thereof;
about 10 to about 50 mEq/L of potassium;
about 1 to about 10 mEq/L of magnesium;
about 2 to about 20 mg/L of zinc;
about 10 to about 200 μg/L of selenium;
about 1 to about 10 mg/L of copper;
about 100 to about 20000 IU of vitamin A;
about 5 to about 4000 IU of vitamin E;
about 200 to about 20000 IU of vitamin D; and
one or more sweeteners.

35. A method for treating or preventing dehydration in a subject in need thereof, the method comprising administering to the subject a composition.

36. The method of claim 35, wherein the subject is a child.

37. (canceled)

38. The method of claim 35, wherein the subject has short bowel syndrome.

39. The method of claim 35, wherein the subject has one or more of diarrhea, vomiting, a viral infection, a bacterial infection, Crohn's disease, ischemic bowel disease, and cancer.

40.-45. (canceled)
